SEQUENCE LISTING (3 sequences)

SEQ ID NO: 1 - DNA, 27 bp, synthetic construct
  source 1..27: mol_type = other DNA
ccccctcgagtggacaatcacaatggg

SEQ ID NO: 2 - DNA, 34 bp, synthetic construct
  source 1..34: mol_type = other DNA
taaggagatatcttaattttcaagctgttcgtta

SEQ ID NO: 3 - protein, 102 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLEN

(19) United States
(12) Patent Application Publication
(10) Pub. No.: US 2025/0018063 A1
(43) Pub. Date: Jan. 16, 2025

(54) Method of Treating Geographic Atrophy with a Gene Therapy Vector Expressing Soluble CD59

(71) Applicant: Janssen Biotech, Inc., Horsham, PA (US)

(72) Inventor: Adam Rogers, Waltham, MA (US)

(21) Appl. No.: 18/711,131

(22) PCT Filed: Nov. 18, 2022
(86) PCT No.: PCT/IB2022/061158

§ 371 Date: May 17, 2024

Related U.S. Application Data
(60) Provisional application No. 63/281,190, filed on Nov. 19, 2021.

(51) Int. Cl. A61K 48/00; A61K 38/17; C12N 15/86

(52) U.S. Cl. CPC A61K 48/0075; A61K 38/177; A61K 48/005; C12N 15/86; C12N 2750/14143

(57) ABSTRACT

The described invention provides method for treating a complement disorder such as age-related macular degeneration (AMD) in a subject, comprising administering a pharmaceutical composition into an affected eye of a subject by ocular injection, wherein the composition comprises a nucleic acid encoding a soluble CD59 (sCD59) protein operably linked to a promoter, wherein the nucleic acid encoding sCD59 is packaged into a delivery vector and wherein the administering results in expression and secretion of the sCD59 protein by cells of the affected eye and the expression results in treatment of affected cells in the affected eye.

### CROSS REFERENCE TO RELATED APPLICATIONS

This application claims the benefit of priority of U.S. Provisional Application No. 63/281,190, filed on Nov. 19, 2021, which is incorporated by reference herein, in its entirety and for all purposes.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “JBI6660WOPCT1_SL.xml”, was created on Nov. 9, 2022 and is 3,843 bytes in size.

### FIELD OF THE INVENTION

The described invention generally relates to the use of gene therapy to treat retinal disease, including age-related macular degeneration (AMD) and geographic atrophy (GA).

### BACKGROUND OF THE INVENTION

Age-related macular degeneration (AMD) is a slow and progressive disease of the macula. AMD is the leading cause of blindness in patients over 60 years of age in developed countries (Friedman, D S, et al. Prevalence of age-related macular degeneration in the United States. Arch Ophthalmol. 2004; 122:564-572; van Leeuwen, R, Klaver, CC, Vingerling, JR, Hofman, A, de Jong, PT. Epidemiology of age-related maculopathy: a review. Eur J Epidemiol. 2003; 18:845-854). Globally, AMD accounts for approximately 9% of all blindness and is predicted to affect approximately 196 million people by 2020 (Wong W L, Su X, Li X, et al. Global prevalence of age-related macular degeneration and disease burden projection for 2020 and 2040: a systematic review and meta-analysis. Lancet Glob Health. 2014 February; 2(2):e106-116).

The most significant loss of vision occurs in advanced AMD. Advanced AMD is divided into 2 categories: (1) ‘wet’ or exudative AMD; and (2) “dry” AMD, also referred to as geographic atrophy (GA). Wet AMD is associated with choroidal neovascularization (CNV) and can be successfully treated using Food and Drug Administration (FDA)-approved vascular endothelial growth factor (VEGF) inhibitors such as ranibizumab or aflibercept. Conversely, there is currently no FDA-approved treatment for GA. Established risk factors that contribute towards AMD include age, smoking, diet, as well as genetic risk factors (van Leeuwen, R, Klaver, C C, Vingerling, J R, Hofman, A, de Jong, P T. Epidemiology of age-related maculopathy: a review. Eur J Epidemiol. 2003; 18:845-854; de Jong, PT. Age-related macular degeneration. N Engl J Med. 2006; 355:1474-1485). Patients suffering from AMD have morbidities beyond vision loss including an increased incidence of depression and anxiety, impaired mobility, and isolation (Dawson S R, Mallen C D, Gouldstone M B, Yarham R, Mansell G. The prevalence of anxiety and depression in people with age-related macular degeneration: a systematic review of observational study data. BMC Ophthalmol. 2014 Jun. 12; 14:78).

Human genetic studies have suggested that overactivation of the complement system may play a role in the pathogenesis of AMD. Single nucleotide polymorphisms in multiple components of the complement pathway including complement factor H, complement factor I, Factor B, and C3 have been associated with increased risk of advanced AMD (Schramm E C, Clark S J, Triebwasser M P, Raychaudhuri S, Seddon J, Atkinson J P. Genetic variants in the complement system predisposing to age-related macular degeneration: a review. Mol Immunol. 2014 October; 61(2):118-125). The complement pathway is a key part of innate immunity in recognizing and killing foreign pathogens and can be activated by the classical, alternative or lectin pathway. The terminal component of the complement pathway is formation of the membrane attack complex (MAC), a complex of proteins on the cell membrane which has cytolytic functions. Multiple lines of evidence suggest that the MAC complex is involved in the pathogenesis of AMD including 1) MAC deposition has been found to be increased in AMD patient samples and 2) individuals with a R95X nonsense mutation in C9, a key component of the MAC complex, results in a 4.7-fold decreased risk of wet AMD (Nichiguchi K M, Yasuma T R, Tomida D, et al. C9-R95X polymorphism in patients with neovascular age-related macular degeneration. Invest Ophthalmol Vis Sci. 2012 Jan. 31; 53(1):508-512).

Thus, preventing formation of the MAC complex may serve as a therapeutic strategy for AMD. A natural inhibitor of the MAC complex is CD59, a glycosylphosphatidylinositol (GPI) anchored membrane bound protein which prevents formation of the MAC complex. Preclinical studies have shown that intravitreal injection of an AAV2 viral vector expressing a soluble form of CD59 (sCD59), could decrease MAC deposition and demonstrated efficacy in a rodent model of wet AMD (Cashman S M, Ramo K, Kumar-Singh R. A non membrane-targeted human soluble CD59 attenuates choroidal neovascularization in a model of age related macular degeneration. PLoS One. 2011 Apr. 28; 6(4):e19078) suggesting that expression of sCD59 can decrease MAC deposition in vivo, and may have therapeutic efficacy in AMD.

### SUMMARY OF THE INVENTION

According to one aspect, the described invention provides a method for treating age-related macular degeneration (AMD) in a subject, the method comprising administering a pharmaceutical composition into an AMD-affected eye of a subject by ocular injection, wherein the composition comprises a nucleic acid encoding a soluble CD59 (sCD59) protein operably linked to a promoter, wherein the nucleic acid encoding sCD59 is packaged into a delivery vector and wherein the administering results in expression and secretion of the sCD59 protein by cells of the AMD-affected eye and the expression results in treatment of AMD-affected cells in the AMD-affected eye.

According to one embodiment, the AMD is geographic atrophy (GA).

According to one embodiment, the ocular injection is an intravitreal injection. According to another embodiment, the intravitreal injection is a single injection.

According to one embodiment, the delivery vector is an adeno-associated virus (AAV) vector. According to another embodiment, the AAV vector is AAV2.

According to one embodiment, the promoter is a CAG promoter.

According to one embodiment, the pharmaceutical composition comprises a dose of viral particles selected from the group consisting of about 3.56×10¹⁰ DNAse-resistant particles (DRP), about 1.071×10¹¹ DRP, about 3.56×10¹¹ DRP and about 1.07×10¹² DRP.

According to another aspect, the described invention provides a method of regulating a complement activity disorder in a subject, the method comprising contacting an affected cell of the subject with a pharmaceutical composition comprising a vector carrying a nucleotide sequence encoding a recombinantly engineered human soluble CD59 (sCD59) protein operably linked to a promoter sequence causing expression of the protein in the affected cell, wherein the sCD59 protein comprises at least one mutation resulting in loss of function of glycosylphosphatidylinositol (GPI) anchoring domain resulting in loss of membrane targeting and observing a physiological indicium of the complement activity disorder after the contacting, in comparison to an abnormal amount of the physiological indicium observed prior to the contacting, wherein a decrease after the contacting compared prior to the contacting is a positive indication that the affected cell is treated.

According to one embodiment, the complement activity disorder is GA.

According to one embodiment, the contacting is by intravitreal injection. According to another embodiment, the intravitreal injection is a single injection.

According to one embodiment, the affected cell is a retinal cell.

According to one embodiment, the vector is AAV2.

According to one embodiment, the physiological indicium is best corrected visual acuity (BCVA). According to another embodiment, the BCVA is measured as mean change from baseline. According to another embodiment, the mean change from baseline is −7.100 letters.

According to one embodiment, the pharmaceutical composition comprises a dose of viral particles selected from the group consisting of about 3.56×10¹⁰ DNAse-resistant particles (DRP), about 1.071×10¹¹ DRP, about 3.56×10¹¹ DRP and about 1.07×10¹² DRP.

According to another aspect, the described invention provides a method of treating a complement disorder comprising contacting a cell with a therapeutically effective amount of a pharmaceutical composition having as an active agent a nucleic acid encoding a human sCD59 protein or a source of expression of a human sCD59 protein comprising administering the pharmaceutical composition to a subject in need thereof.

According to one embodiment, the complement disorder is GA.

According to one embodiment, the contacting is by intravitreal injection. According to another embodiment, the intravitreal injection is a single injection.

According to one embodiment, the affected cell is a retinal cell.

According to one embodiment, the therapeutically effective amount is a dose of viral particles selected from the group consisting of about 3.56×10¹⁰ DNAse-resistant particles (DRP), about 1.071×10¹¹ DRP, about 3.56×10¹¹ DRP and about 1.07×10¹² DRP.

### BRIEF DESCRIPTION OF THE DRAWINGS

FIG. 1 shows a schematic diagram depicting a dose-escalation study conducted to establish the safety of a single intravitreal injection of gene therapy vector AAVCAGsCD59 for the treatment of patients with advanced dry age-related macular degeneration (AMD) with geographic atrophy (GA). DRP=DNAse-resistant particles.

FIG. 2 shows a Spaghetti Plot depicting Change from Baseline to selected visits in Square root transformed of geographic atrophy (GA) lesion area (mm) in the Study Eye, labeled by Cohort. sqrt=square root.

FIG. 3 shows a plot depicting least square (LS) mean with 95% confidence interval (CI) for Change from Baseline in Square root transformed of geographic atrophy (GA) lesion area (mm) measured by fundus autofluorescence (FAF) over time in Study Eye, labeled by Cohort. CHG=change; sqrt=square root.

FIG. 4 shows a plot depicting least square (LS) mean with 95% confidence interval (CI) for Change from Baseline in Square root transformed of geographic atrophy (GA) lesion area (mm) measured by fundus autofluorescence (FAF) over time in Study Eye, (Pooled Cohort). CHG=change; sqrt=square root.

FIG. 5 shows a plot depicting least square (LS) mean with 95% confidence interval (CI) for Change from Baseline in geographic atrophy (GA) lesion area (mm²) measured by fundus autofluorescence (FAF) over time in Study Eye, labelled by Cohort.

FIG. 6 shows a plot depicting least square (LS) mean with 95% confidence interval (CI) for Change from Baseline in geographic atrophy (GA) lesion area (mm²) measured by fundus autofluorescence (FAF) over time in Study Eye, (Pooled Cohort).

FIG. 7 shows a Spaghetti Plot Change from Baseline to selected visits in Distance best corrected visual acuity (BCVA) (letters) in the Study Eye, labeled by Cohort; Full Analysis Set. CHG=change.

FIG. 8 shows a Spaghetti Plot Change from Baseline to selected visits in Distance best corrected visual acuity (BCVA) (letters) in the Fellow Eye, labelled by Cohort; Full Analysis Set. CHG=change.

### DETAILED DESCRIPTION OF THE INVENTION

The described invention can be better understood from the following description of exemplary embodiments, taken in conjunction with the accompanying figures and drawings. It should be apparent to those skilled in the art that the described embodiments provided herein are merely exemplary and illustrative and not limiting.

### Definitions

Various terms used throughout this specification shall have the definitions set out herein.

The term “administering” as used herein includes in vivo administration, as well as administration directly to tissue ex vivo. Generally, compositions can be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or can be locally administered by means such as, but not limited to, injection, implantation, grafting, topical application, or parenterally.

The term “attenuate” as used herein means to reduce the force, effect, or value of.

The term “CD59” as used herein refers to a membrane-bound glycoprotein found associated with membranes of cells including both human hematopoietic and non-hematopoietic cells, for example on endothelial cells, peripheral nerve fibers, neurons, microglia, oligodendrocytes, astrocytes, ependymal cells, epithelial cells, acinar cells of the salivary glands, bronchial epithelium, renal tubules and squamous epithelium. CD59 protein inhibits assembly of functional membrane attack complexes (MACs) and thus protects cells from complement-mediated activation and/or lysis. The protein structure of CD59 includes a single cysteine-rich domain, a hydrophobic core with three loops and a small fourth helical loop (Yu et al. 1997 Journal of Experimental Medicine 185(4): 745-753). Human CD59 includes 26 amino acids located at the C terminus, which specifies a signal sequence for attachment of a glycosyl phosphatidyl inositol anchor (GPI anchor) at amino acid asparagine at position 77. A cDNA sequence of CD59 is shown in Fodor et al., U.S. Pat. No. 5,624,837 issued Apr. 29, 1997, which is incorporated herein by reference in its entirety.

The term “condition”, as used herein refers to a variety of health states and is meant to include disorders or diseases caused by any underlying mechanism or injury.

The term “disease” or “disorder,” as used herein refers to an impairment of health or a condition of abnormal functioning.

The term “dosage unit form” as used herein refers to a physically discrete unit of active agent appropriate for the patient to be treated.

The term “drug” as used herein refers to a therapeutic agent or any substance used in the prevention, diagnosis, alleviation, treatment, or cure of disease.

The term “drusen” as used herein refers to yellow deposits under the retina.

The term “enhance” as used herein in its various grammatical forms refers to an increase or to intensify in quality or quantity, or to make better or augment.

The terms “functional equivalent” or “functionally equivalent” are used interchangeably herein to refer to substances, molecules, polynucleotides, proteins, peptides, or polypeptides having similar or identical effects or use. A polypeptide functionally equivalent to SEQ ID NO: 3, for example, may have a biologic activity, e.g., an inhibitory activity, kinetic parameters, salt inhibition, a cofactor-dependent activity, and/or a functional unit size that is substantially similar or identical to the expressed polypeptide of SEQ ID NO: 3.

The term “inhibit” and its various grammatical forms, including, but not limited to, “inhibiting” or “inhibition”, are used herein to refer to reducing the amount or rate of a process, to stopping the process entirely, or to decreasing, limiting, or blocking the action or function thereof. Inhibition can include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. Enzyme inhibitors are molecules that bind to enzymes thereby decreasing enzyme activity. The binding of an inhibitor can stop a substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injury,” as used herein refers to damage or harm to a structure or function of the body caused by an outside agent or force, which can be physical or chemical.

The terms “membrane attack complex” and “MAC” are used interchangeably herein to refer to an effector of the immune system comprising a complex of proteins typically formed on the surface of pathogen cell membranes as a result of activation of a host's complement system. Antibody-mediated complement activation leads to MAC deposition on the surface of infected cells, leading to pores that disrupt the cell membrane of the infected cells, resulting in cell lysis and death. The MAC is composed of complement components C5b, C6, C7, C8 and several C9 molecules.

The term “modify” as used herein means to change, vary, adjust, temper, alter, affect or regulate to a certain measure or proportion in one or more particulars.

The term “modulate” as used herein means to regulate, alter, adapt, or adjust to a certain measure or proportion.

The term “nucleic acid” is used herein to refer to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” is used herein to refer to a chemical compound that consists of a heterocyclic base, a sugar, and one or more phosphate groups. In the most common nucleotides, the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. Purines include adenine (A), and guanine (G); pyrimidines include cytosine (C), thymine (T), and uracil (U).

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity.”

(a) The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

(b) The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences, 8:155-65 (1992), and Pearson, et al., Methods in Molecular Biology, 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination.

(c) The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e) The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid. The term “substantial identity” of protein sequences refers to a first amino acid sequence that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 60% identity, or at least 75%, 80%, 85%, 90%, 95%, 96%, 98%, or 99% identity.

The term “parenteral” as used herein refers to introduction into the body by way of an injection (i.e., administration by injection), including, for example, intraocularly (also known as intravitreally) (i.e., an injection into the vitreous of the eye), subretinally (i.e., an injection into the subretinal space which exists between the photoreceptors of the retina and the retinal pigment epithelium (RPE) layer), subcutaneously (i.e., an injection beneath the skin), intramuscularly (i.e., an injection into a muscle); intravenously (i.e., an injection into a vein), intrathecally (i.e., an injection into the space around the spinal cord), intrasternal injection, or infusion techniques. A parenterally administered composition of the described invention is delivered using a needle, e.g., a surgical needle. The term “surgical needle” as used herein, refers to any needle adapted for delivery of fluid (i.e., capable of flow) compositions of the described invention into a selected anatomical structure. Injectable preparations, such as sterile injectable aqueous or oleaginous suspensions, can be formulated according to the known art using suitable dispersing or wetting agents and suspending agents.

As used herein the term “pharmaceutically acceptable carrier” refers to any substantially non-toxic carrier conventionally useable for administration of pharmaceuticals in which the isolated polypeptide of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

The term “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids.

The terms “polypeptide” and “protein” also are used herein in their broadest sense to refer to a sequence of subunit amino acids, amino acid analogs, or peptidomimetics. The subunits are linked by peptide bonds, except where noted. The polypeptides described herein may be chemically synthesized or recombinantly expressed. Polypeptides of the described invention also can be synthesized chemically. Synthetic polypeptides, prepared using the well-known techniques of solid phase, liquid phase, or peptide condensation techniques, or any combination thereof, can include natural and unnatural amino acids. Amino acids used for peptide synthesis may be standard Boc (N-α-amino protected N-α-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). Both Fmoc and Boc N-α-amino protected amino acids can be obtained from Sigma, Cambridge Research Biochemical, or other chemical companies familiar to those skilled in the art. In addition, the polypeptides can be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R1-CH₂—NH—R2, where R1 and R2 are amino acid residues or sequences. A reduced peptide bond may be introduced as a dipeptide subunit. Such a polypeptide would be resistant to protease activity, and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, the protein is specifically reactive to antibodies elicited to the same protein but consisting entirely of naturally occurring amino acids.

The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. In some embodiments, the peptide is of any length or size.

The terms “preserve”, “preserved”, “preserving” or “preservation” as used herein refer to maintaining, keeping safe from harm or injury, protecting, sparing or maintaining function.

The terms “prevent”, “prevented”, “preventing” or “prevention” as used herein refer to the keeping, hindering or averting of an event, act, or action from happening, occurring or arising.

The term “recombinant” refers to a cell or vector that has been modified by the introduction of a heterologous nucleic acid or the cell that is derived from a cell so modified. Recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation transduction/transposition) such as those occurring without deliberate human intervention.

The term “recombinant expression cassette” refers to a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, virus, or nucleic acid fragment. Typically, the recombinant expression cassette portion of an expression vector includes, among other sequences, a nucleic acid to be transcribed, a promoter, and a transcription termination signal such as a poly-A signal.

The term “recombinant host” refers to any prokaryotic or eukaryotic cell that contains either a cloning vector or an expression vector. This term also includes those prokaryotic or eukaryotic cells that have been genetically engineered to contain the cloned genes, or gene of interest, in the chromosome or genome of the host cell.

The term “recombinant protein” as used herein refers to a protein produced by genetic engineering, for example, by manipulation of genetically modified organisms such as micro-organisms.

The term “reduce” or “reducing” as used herein refers to the limiting of an occurrence of a disorder in individuals at risk of developing the disorder.

The term “regulate” as used herein means to control or maintain a process, function or mechanism, for example, a biological process.

The term “similar” is used interchangeably with the terms analogous, comparable, or resembling, meaning having traits or characteristics in common.

The term “solution” as used herein refers to a homogeneous mixture of two or more substances. It is frequently, though not necessarily, a liquid. In a solution, the molecules of the solute (or dissolved substance) are uniformly distributed among those of the solvent.

The terms “soluble CD59”, “sCD59” and “membrane independent CD59” as used herein refer to a CD59 amino acid sequence that lacks a glycosylphosphatidylinositol (GPI) anchor or has a modified GPI anchor that lacks function and ability to bind to a cell membrane or a cell-membrane-associated structure such as a membrane-bound protein.

The term “stimulate” in any of its grammatical forms as used herein refers to inducing activation or increasing activity.

The term “suspension” as used herein refers to a dispersion (mixture) in which a finely-divided species is combined with another species, with the former being so finely divided and mixed that it doesn't rapidly settle out. In everyday life, the most common suspensions are those of solids in liquid.

As used herein, the terms “subject” or “individual” or “patient” or “participant” are used interchangeably to refer to a member of an animal species of mammalian origin, including humans. The term “a subject in need thereof” is used to refer to a subject having, or at risk of progression to heart failure, including a subject having an AMI that leads to a disease manifestation of left ventricular remodeling.

The phrase “subject in need of such treatment” as used herein refers to a patient who suffers from a disease, disorder, condition, or pathological process. In some embodiments, the term “subject in need of such treatment” also is used to refer to a patient who (i) will be administered at least one dose of the adenovirus vector construct expressing human soluble CD59 of the described invention; (ii) is receiving at least one dose of the adenovirus vector construct expressing human soluble CD59 of the described invention; or (iii) has received at least one dose of the adenovirus vector construct expressing human soluble CD59 of the described invention, unless the context and usage of the phrase indicates otherwise.

The term “substantially similar” as used herein means that a first value, aspect, trait, feature, number, or amount is of at least 70%, at least 75%, at least 80%, at least 85%, at least 90%, or at least 95% of a second value, aspect, trait, feature, number, or amount. For example, a polypeptide substantially similar to (SEQ ID NO: 3) would have at least 70% amino acid sequence identity, at least 75% amino acid sequence identity, at least 80% amino acid sequence identity, at least 90% sequence identity, or at least 95% amino acid sequence identity to amino acid sequence (SEQ ID NO: 3).

The term “substitution” is used herein to refer to a situation in which a base or bases are exchanged for another base or bases in a DNA sequence. Substitutions may be synonymous substitutions or nonsynonymous substitutions. As used herein, “synonymous substitutions” refer to substitutions of one base for another in an exon of a gene coding for a protein, such that the amino acid sequence produced is not modified. The term “nonsynonymous substitutions” as used herein refer to substitutions of one base for another in an exon of a gene coding for a protein, such that the amino acid sequence produced is modified.

The term “symptom” as used herein refers to a phenomenon that arises from and accompanies a particular disease or disorder and serves as an indication of it.

The term “syndrome,” as used herein, refers to a pattern of symptoms indicative of some disease or condition.

The term “therapeutic agent” as used herein refers to a drug, molecule, nucleic acid, protein, metabolite, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The terms “therapeutic amount”, “therapeutically effective amount”, an “amount effective”, or “pharmaceutically effective amount” of an active agent is used interchangeably to refer to an amount that is sufficient to provide the intended benefit of treatment. An effective amount of the active agent(s) that can be employed according to the described invention generally ranges from about 1×10¹⁰ DNAse-resistant particles (DRP) to 1×10¹² DRP per dose. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amount” and “pharmaceutically effective amount” includes prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

The term “therapeutic effect” as used herein refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest, reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The term “therapeutic window” refers to a concentration range that provides therapeutic efficacy without unacceptable toxicity. Following administration of a dose of a drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

Instead, another dose of drug should be given to maintain concentrations within the therapeutic window. In general, the lower limit of the therapeutic range of a drug appears to be approximately equal to the drug concentration that produces about half of the greatest possible therapeutic effect, and the upper limit of the therapeutic range is such that no more than about 5% to about 10% of patients will experience a toxic effect. These figures can be highly variable, and some patients may benefit greatly from drug concentrations that exceed the therapeutic range, while others may suffer significant toxicity at much lower values. The therapeutic goal is to maintain steady-state drug levels within the therapeutic window. For most drugs, the actual concentrations associated with this desired range are not and need not be known, and it is sufficient to understand that efficacy and toxicity are generally concentration-dependent, and how drug dosage and frequency of administration affect the drug level. For a small number of drugs where there is a small (two- to three-fold) difference between concentrations resulting in efficacy and toxicity, a plasma-concentration range associated with effective therapy has been defined.

In cases where a target level strategy is reasonable, wherein a desired target steady-state concentration of the drug (usually in plasma) associated with efficacy and minimal toxicity is chosen, and a dosage is computed that is expected to achieve this value. Drug concentrations subsequently are measured and dosage is adjusted if necessary to approximate the target more closely.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The terms “variants”, “mutants”, and “derivatives” are used herein to refer to nucleotide or polypeptide sequences with substantial identity to a reference nucleotide or polypeptide sequence. The differences in the sequences may be the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce polypeptide variants of polypeptide SEQ ID NO: 3 having single or multiple amino acid substitutions, deletions, additions or replacements, but functionally equivalent to SEQ ID NO: 3. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, for example, an epitope for an antibody. The techniques for obtaining such variants, including, but not limited to, genetic (suppressions, deletions, mutations, etc.), chemical, and enzymatic techniques, are known to the skilled artisan. As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “vector” as used herein refers to a carrier that is genetically engineered to deliver a gene to a cell. The term “viral vector” as used herein refers to a virus used as a vector to deliver a gene of interest by infecting a cell. Such viruses are modified so they cannot cause disease when used in humans. Types of viruses include, but are not limited to, retroviruses, which integrate their genetic material (including the gene interest) into a chromosome in a cell, and adenoviruses, which introduce their DNA (including the gene of interest) into the nucleus of a cell without integrating the genetic material into a chromosome.

The term “vehicle” as used herein refers to a substance that facilitates the use of a drug or other material that is mixed with it.

According to some embodiments, the described invention provides a nucleotide sequence encoding human soluble CD59 (sCD59) protein. According to some embodiments, the nucleotide sequence is a complementary DNA (cDNA) sequence.

cDNA sequences encoding human CD59 are known in the art. For example, cDNA sequences have been reported by Sawada, R. et al. 1989 Nucleic Acids Res 17(16): 6728 and are available from the American Type Tissue Culture Collection (ATCC, Manassas, Va.). A cDNA encoding CD59 has also been cloned from human T-cell leukemia (YT) and human erythroleukemia (K562) cell lines, and CD59 has been transiently expressed in COS cells (Walsh, L. A. et al. 1990 Eur J. Immol 21(3): 847-850).

According to some embodiments, the human sCD59 lacks the primary amino acid sequence for a functional glycosylphosphatidylinositol (GPI) anchor. According to some embodiments, the human sCD59 comprises a modified GPI anchor domain amino acid sequence that is functionally defective and lacks the ability to target a membrane. According to some embodiments, the modified GPI anchor domain amino acid sequence comprises a variation. Such variations include, but are not limited to, substitution and deletion of nucleic acids encoding amino acids at omega positions used to reduce or eliminate the attachment of the GPI anchor or reduce or eliminate the effective functionality of the GPI anchor. Omega amino acids are amino acids to which GPI is transferred. For example, such a variation includes, but is not limited to, substituting the nucleic acids encoding hydrophobic leucine (e.g., nucleic acids CTG) and alanine (e.g., nucleic acids GCA) with nucleic acids encoding glycine (e.g., nucleic acids CAG) and glutamate (e.g., nucleic acids GAA), which are less hydrophobic (i.e., more hydrophilic) amino acids. Alternatively, a variation may include substituting the omega residue with another amino acid, such as a glycine for a tyrosine.

According to some embodiments, the human sCD59 protein of the described invention includes conservative sequence modifications. Conservative sequence modifications are amino acid modifications that do not significantly affect or alter the characteristics of the human sCD59 protein containing the amino acid sequence, i.e., amino acid sequences of sCD59 that present these side chains at the same relative positions will function in a manner similar to human sCD59. Such conservative modifications include amino acid substitutions, additions and deletions. Methods of modifying amino acid sequences are known in the art e.g., site-directed mutagenesis or PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. According to some embodiments, the human sCD59 protein of the described invention comprises conservative amino acid substitutions. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

According to some embodiments, the human sCD59 amino acid sequence is an amino acid sequence that is substantially identical to that of the wild-type sequence. According to some embodiments, the human sCD59 amino acid sequence is at least 70% identical to that of the wild-type sequence. According to some embodiments, the human sCD59 amino acid sequence is at least 75% identical to that of the wild-type sequence. According to some embodiments, the human sCD59 amino acid sequence is at least 80% identical to that of the wild-type sequence. According to some embodiments, the human sCD59 amino acid sequence is at least 90% identical to that of the wild-type sequence. According to some embodiments, the human sCD59 amino acid sequence is at least 95% identical to that of the wild-type sequence.

According to some embodiments, the human sCD59 comprises SEQ ID NO: 3. According to some embodiments, the human sCD59 consists essentially of SEQ ID NO: 3. According to some embodiments, the human sCD59 is SEQ ID NO: 3.

According to some embodiments, the human sCD59 of the described invention is a recombinant protein.

A variety of commercially available expression vector/host systems are useful to contain and express a CD59 protein encoding sequence. These include but are not limited to microorganisms such as bacteria transformed with recombinant bacteriophage, plasmid or cosmid DNA expression vectors; yeast transformed with yeast expression vectors; insect cell systems contacted with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems. See Ausubel et al., Current Protocols in Molecular Biology, John Wiley & Sons, New York, N.Y., 1989.

Techniques for altering a nucleic acid sequence to produce a recombinant protein are well known in the art of genetics and molecular biology. Traditional strategies for recombinant protein expression involve transfecting cells with a DNA vector that contains a template for expressing a desired protein and then culturing the cells so that they transcribe and translate the desired protein. The cells are then lysed to extract the expressed protein for subsequent purification.

Types of cells used in recombinant protein expression include, but are not limited to, prokaryotic and eukaryotic cells. Prokaryotic cells include, but are not limited to, bacterial cells. A non-limiting example of a bacterial cell includes Escherichia coli. Eukaryotic cells include, but are not limited to, mammalian, insect, yeast and algae cells. Non-limiting examples of mammalian cells include human embryonic kidney cells (e.g., HEK293, HEK293T), baby hamster kidney cells (e.g., BHK21), Chinese hamster ovary (CHO) cells, mouse myeloma cells (e.g., NS0) and murine non-producing hybridoma cells (e.g., SP2/O—Ag14). Non-limiting examples of insect cells include, Spodoptera frugiperda pupa ovarian cells (e.g., Sf9, Sf21). A non-limiting example of a yeast cell includes Saccharomyces cerevisiae. A non-limiting example of an algae cell includes Chlamydomonas reinhardtii.

Methods for expressing recombinant proteins also include cell-free systems. Cell-free protein expression includes the in vitro production of recombinant proteins in solution (i.e., cell lysate) using biomolecular translation machinery extracted from cells.

Various methods of protein purification may be employed and such methods are known in the art and described, for example, in Deutscher, Methods in Enzymology, 182 (1990); Scopes, Protein Purification: Principles and Practice, Springer-Verlag, New York (1982). The purification step(s) selected will depend, for example, on the nature of the production process used and the particular protein produced.

According to some embodiments, the human sCD59 is a synthetic protein. Methods of preparing synthetic proteins are well-known in the art. See, for example, Peptide Synthesis Protocols, Methods in Molecular Biology, vol. 35, Pennington, M. W. and Dunn, B. M., 1995, XII, Humana Press, Inc. Totowa, New Jersey. Synthetic proteins, prepared using well-known techniques such as solid phase, liquid phase, or peptide condensation techniques, or any combination thereof, can include natural and unnatural amino acids. Amino acids used for peptide synthesis may be standard Boc amino acid resin with the standard deprotecting, neutralization, coupling and wash protocols of original solid phase procedure of Merrifield (1963, J. Am. Chem. Soc. 85:2149-2154), or the base-labile N-α-amino protected 9-Fluorenylmethoxycarbonyl (Fmoc) amino acids first described by Carpino and Han (1972, J. Org. Chem. 37:3403-3409). Both Boc and Fmoc amino protected amino acids can be obtained from Sigma or other chemical companies familiar to those skilled in art. The peptides can also be synthesized with other N-α protecting groups familiar to those skilled in the art.

According to some embodiments, the nucleotide sequence encoding human sCD59 is used to construct an expression vector. According to some embodiments, the described invention provides a human sCD59 expression construct. Methods used to construct expression vectors are well known to those skilled in the art. For example, such methods can be used to construct expression vectors containing the nucleotide sequence encoding the human sCD59 protein operably linked to appropriate transcriptional and translational control elements. These methods include, but are not limited to, in vitro recombinant DNA techniques, synthetic techniques and in vivo recombination or genetic recombination. Such techniques are described in Sambrook et al., Molecular Cloning: A Laboratory Manual, Cold Spring Harbor Press, Plainview, N.Y., 1989.

According to some embodiments, the nucleotide sequence encoding human sCD59 is operably linked to a promoter. According to some embodiments, the promoter is a constitutive promoter. According to some embodiments, the promoter is a cell cycle-specific promoter. According to some embodiments, the promoter is a ubiquitous promoter. According to some embodiments, the promoter is a tissue-specific promoters. Examples of tissue-specific promoters include, but are not limited to, human rhodopsin kinase (hRK) promoter and retinal pigmemt epithelium specific promoter (e.g., RPE65 promoter). According to some embodiments, the promoter is a metabolically regulated promoter. According to some embodiments, the promoter is an inducible promoter. According to some embodiments, the promote is a hybrid promoter. A non-limiting example of a hybrid promoter is cytomegalovirus (CMV) early enhancer element/the first exon and the first intron of chicken beta-actin gene/the splice acceptor of the rabbit beta-globin gene (CAG). Non-limiting examples of promoters are shown in Evans et al. U.S. Pat. No. 6,677,311 B1 issued Jan. 13, 2004; Clark et al. U.S. Pat. No. 7,109,029 B2 issued Sep. 19, 2006; and Hallenbeck et al. U.S. Pat. No. 5,998,205 issued Dec. 7, 1999, each of which is incorporated herein by reference in its entirety.

According to some embodiments, the nucleotide sequence encoding human sCD59 operably linked to a promoter is packaged into a delivery vector. According to some embodiments, the human sCD59 expression construct is packaged into a delivery vector.

According to some embodiments, the delivery vector is a virus vector. Virus vectors include, but are not limited to, adenovirus vectors, lentivirus vectors, adeno-associated virus (AAV) vectors, and helper-dependent adenovirus vectors.

Adenovirus vectors are commercially available from American Type Tissue Culture Collection (Manassas, Va.). Methods of constructing adenovirus vectors and using adenovirus vectors are described in Klein et al. 2007 Ophthalmology 114: 253-262, and van Lecuwen et al. 2003 Eur. J. Epidemiol. 18: 845-854. Adenovirus vectors have been used in eukaryotic gene expression (Levrero et al. 1991 Gene, 101: 195-202) and vaccine development (Graham et al. 1991 Methods in Molecular Biology: Gene Transfer and Expression Protocols 7, (Murray, Ed.), Humana Press, Clifton, N.J., 109-128). Further, recombinant adenovirus vectors are used for gene therapy (Wu et al. U.S. Pat. No. 7,235,391 issued Jun. 26, 2007 which is incorporated herein by reference in its entirety).

Recombinant adenovirus vectors are generated, for example, from homologous recombination between a shuttle vector and a provirus vector (Wu et al., U.S. Pat. No. 7,235,391 issued Jun. 26, 2007). The adenovirus vectors used herein are replication defective. For example, the adenovirus vectors are conditionally defective, lacking adenovirus E1 region. A polynucleotide encoding a protein of interest, human sCD59 for example, is introduced at the position from which the E1-coding sequences have been removed. Alternatively, the polynucleotide encoding the protein of interest (e.g., human sCD59) may be inserted in the E3 region of the adenovirus.

Defective adenovirus vectors can be generated and propagated using a helper cell line. Helper cell lines may be derived from human cells such as, 293 human embryonic kidney cells (HEK293), muscle cells, hematopoietic cells or other human embryonic mesenchymal or epithelial cells. Alternatively, the helper cells may be derived from the cells of other mammalian species that are permissive for human adenovirus, e.g., Vero cells or other monkey embryonic mesenchymal or epithelial cells. Generation and propagation of these replication defective adenovirus vectors using a helper cell line is described in Graham et al 1977 J. Gen. Virol. 36: 59-72.

Lentiviral packaging vectors are commercially available from Invitrogen Corporation (Carlsbad Calif.). An HIV-based packaging system for the production of lentiviral vectors is prepared using constructs described in Naldini et al. 1996 Science 272: 263-267; Zufferey et al. 1997 Nature Biotechnol. 15: 871-875; and Dull et al. 1998 J. Virol. 72: 8463-8471. A number of vector constructs are available to be packaged using a system, based on third-generation lentiviral SIN vector backbone (Dull et al. 1998 J. Virol. 72: 8463-8471). For example, the vector construct pRRLsinCMVGFPpre contains a 5′ LTR in which the HIV promoter sequence has been replaced with that of Rous sarcoma virus (RSV), a self-inactivating 3′ LTR containing a deletion in the U3 promoter region, the HIV packaging signal, RRE sequences linked to a marker gene cassette consisting of the Aequora jellyfish green fluorescent protein (GFP) driven by the CMV promoter, and the woodchuck hepatitis virus PRE element, which appears to enhance nuclear export. The GFP marker gene allows quantitation of transfection or transduction efficiency by direct observation of UV fluorescence microscopy or flow cytometry (Kafri et al. 1997 Nature Genet. 17: 314-317; and Sakoda et al. 1999 J. Mol. Cell. Cardiol. 31: 2037-2047).

Manipulation of retroviral nucleic acids to construct a retroviral vector containing a gene of interest (e.g., gene that encodes for human sCD59 protein) and packaging cells is accomplished using techniques known in the art (See, e.g., Ausubel, et al., 1992, Volume 1, Section III (units 9.10.1-9.14.3); Sambrook, et al., 1989. Molecular Cloning: A Laboratory Manual. Second Edition. Cold Spring Harbor Laboratory Press, Cold Spring Harbor, N.Y.; Miller, et al., Biotechniques. 7:981-990, 1989; Eglitis, et al., Biotechniques. 6:608-614, 1988; U.S. Pat. Nos. 4,650,764, 4,861,719, 4,980,289, 5,122,767, and 5,124,263; and PCT patent publications numbers WO 85/05629, WO 89/07150, WO 90/02797, WO 90/02806, WO 90/13641, WO 92/05266, WO 92/07943, WO 92/14829, and WO 93/14188, each of which is incorporated by reference in its entirety).

A retroviral vector can be constructed and packaged into non-infectious transducing viral particles (virions) using an amphotropic packaging system. Examples of such packaging systems are described in Miller et al. 1986 Mol. Cell Biol. 6:2895-2902; Markowitz et al. 1988 J. Virol. 62:1120-1124; Cosset et al. 1990 J. Virol. 64: 1070-1078; U.S. Pat. Nos. 4,650,764, 4,861,719, 4,980,289, 5,122,767, and 5,124,263, and PCT patent publications numbers WO 85/05629, WO 89/07150, WO 90/02797, WO 90/02806, WO 90/13641, WO 92/05266, WO 92/07943, WO 92/14829, and WO 93/14188, each of which is incorporated by reference in its entirety. Generation of “producer cells” can be accomplished by introducing retroviral vectors into the packaging cells. Examples of such retroviral vectors are found in, for example, Korman et al. 1987 Proc. Natl. Acad. Sci. USA. 84: 2150-2154; Morgenstern et al. 1990 Nucleic Acids Res. 18: 3587-3596; U.S. Pat. Nos. 4,405,712, 4,980,289, and 5,112,767; and PCT patent publications numbers WO 85/05629, WO 90/02797, and WO 92/07943.

Herpesvirus packaging vectors are commercially available from Invitrogen Corporation, (Carlsbad, Calif.). Exemplary herpesviruses include, but are not limited to, an a-herpesvirus, such as Varicella-Zoster virus or pseudorabies virus; a herpes simplex virus such as HSV-1 or HSV-2; or a herpesvirus such as Epstein-Barr virus. A method for preparing empty herpesvirus particles that can be packaged with a desired nucleotide segment, for example a human sCD59 nucleotide or polynucleotide sequence, in the absence of a helper virus that is capable to most herpesviruses is described in Fraefel et al. (U.S. Pat. No. 5,998,208, issued Dec. 7, 1999 which is incorporated by reference in its entirety).

The herpesvirus DNA vector can be constructed using techniques known to the skilled artisan. For example, DNA segments encoding the entire genome of a herpesvirus is divided among a number of vectors capable of carrying large DNA segments, e.g., cosmids (Evans, et al., Gene 79, 9-20, 1989), yeast artificial chromosomes (YACS) (Sambrook, J. et al., MOLECULAR CLONING: A LABORATORY MANUAL, 2nd Edition, Cold Spring Harbor Press, Cold Spring Harbor, N.Y., 1989) or E. coli F element plasmids (O'Conner et al. 1989 Science 244:1307-1313). For example, sets of cosmids have been isolated which contain overlapping clones that represent the entire genomes of a variety of herpesviruses including Epstein-Barr virus, Varicella-Zoster virus, pseudorabies virus and HSV-1. See M. van Ziji et al. 1988 J. Virol. 62: 2191; Cohen et al. 1993 Proc. Nat'l Acad. Sci. U.S.A. 90: 7376; Tomkinson et al. 1993 J. Virol. 67: 7298; and Cunningham et al. 1993 Virology 197: 116.

Adeno-associated virus (AAV) is a dependent parvovirus in that it depends on co-infection with another virus (either adenovirus or a member of the herpes virus family) to undergo a productive infection in cultured cells (Muzyczka 1992 Curr. Top. Microbiol. Immunol., 158:97 129). For example, recombinant AAV (rAAV) virus can be made by co-transfecting a plasmid containing a gene of interest (e.g., human sCD59 gene), flanked by the two AAV terminal repeats (McLaughlin et al. 1988 J. Virol., 62(6): 1963-1973; Samulski et al. 1989 J. Virol, 63: 3822-3828) and an expression plasmid containing wild-type AAV coding sequences without terminal repeats. Cells are also contacted or transfected with adenovirus or plasmids carrying the adenovirus genes required for AAV helper function.

Unlike most viruses, AAVs are innately nonpathogenic, poorly immunogenic, and broadly tropic, making them attractive gene delivery candidates for virus-based gene therapies. Most naturally occurring AAVs utilize glycan moieties for initial attachment to a cell surface, and these interactions have been well characterized for a number of serotypes. The interacting glycan moieties identified include AAV serotype 2 (AAV2), AAV3, AAV6 and AAV8; N-terminal galactose for AAV9; and specific N- or O-linked sialic acid moieties for AAV1, -4, -5, and -6. Serotypes differ by the types of cells they infect, making AAV a very useful system for preferentially transducing specific cell types.

Adeno-associated virus (AAV) packaging vectors are commercially available from GeneDetect (Auckland, New Zealand). AAV has a broad host range for infectivity (Tratschin et al. 1984 Mol. Cell. Biol. 4: 2072-2081; Laughlin et al. 1986 J. Virol., 60(2): 515-524; Lebkowski et al. 1988 Mol. Cell. Biol. 8(10): 3988-3996; McLaughlin et al. 1988 J. Virol. 62(6):1963-1973).

Methods of constructing AAV vectors and using AAV vectors are known in the art. Such methods are described, for example, in U.S. Pat. No. 5,139,941 (Wu et al.) issued Jun. 26, 2007 and U.S. Pat. No. 4,797,368 (Carter et al.) issued Jan. 10, 1989. Use of AAV in gene delivery is further described in LaFace et al. 1988 Virology 162(2): 483 486; Zhou et al. 1993 Exp. Hematol, 21: 928-933; Flotte et al. 1992 Am. J. Respir. Cell Mol. Biol. 7(3): 349-356; and Walsh et al. 1994 J. Clin. Invest 94: 1440-1448.

Recombinant AAV vectors have been used successfully for in vitro and in vivo transduction of marker genes (Kaplitt et al. 1994 Nat Genet., 8(2):148-154; Lebkowski et al. 1988 Mol. Cell. Biol. 8(10): 3988-3996; Samulski et al. 1991 EMBO J. 10: 3941-3950; Shelling and Smith 1994 Gene Therapy, 1: 165-169; Yoder et al. 1994 Blood, 82 (Supp.): 1: 347A; Zhou et al. 1993 Exp. Hematol 21: 928-933; Tratschin et al. 1985 Mol. Cell. Biol. 5: 3258-3260; McLaughlin et al. 1988 J. Virol. 62(6): 1963-1973) and transduction of genes involved in human diseases (Flotte et al. 1992 Am. J. Respir. Cell Mol. Biol. 7(3): 349-356; Ohi et al. 1990 Gene, 89(2): 279-282; Walsh et al. 1994 J. Clin. Invest. 94: 1440-1448; and Wei et al. 1994 Gene Therapy, 1: 261 268).

According to some embodiments, the nucleotide sequence encoding human sCD59 operably linked to a promoter is packaged into an adeno-associated virus (AAV) vector. According to some embodiments, the AAV vector is AAV2. According to some embodiments, the AAV vector is AAV5. According to some embodiments, the AAV vector is AAV8.

According to some embodiments, the human sCD59 expression vector is packaged into an adeno-associated virus (AAV) vector. According to some embodiments, the AAV vector is AAV2. According to some embodiments, the AAV vector is AAV5. According to some embodiments, the AAV vector is AAV8.

According to some embodiments, the nucleotide sequence encoding human sCD59 is packaged between inverted terminal repeat (ITR) sequences within an AAV vector. According to some embodiments, the nucleotide sequence encoding human sCD59 operably linked to a promoter is packaged between inverted terminal repeat (ITR) sequences within an AAV vector. According to some embodiments, the human sCD59 expression vector is packaged between inverted terminal repeat (ITR) sequences within an AAV vector. According to some embodiments, the ITR sequences are AAV2 sequences. According to some embodiments, the ITR sequences are AAV5 sequences. According to some embodiments the ITR sequences are AAV8 sequences.

According to some embodiments, the AAV vector is a hybrid vector. Hybrid vectors contain ITR sequences from one AAV serotype and a capsid protein from a different AAV serotype. According to some embodiments, the hybrid vector comprises ITR sequences from AAV2 and a capsid protein from AAV5 (AAV2/5). According to some embodiments, the hybrid vector comprises ITR sequences from AAV2 and a capsid protein from AAV8 (AAV2/8). According to some embodiments, the hybrid vector comprises ITR sequences from AAV5 and a capsid protein from AAV2 (AAV5/2). According to some embodiments, the hybrid vector comprises ITR sequences from AAV5 and a capsid protein from AAV8 (AAV5/8). According to some embodiments, the hybrid vector comprises ITR sequences from AAV8 and a capsid protein from AAV2 (AAV8/2). According to some embodiments, the hybrid vector comprises ITR sequences from AAV8 and a capsid protein from AAV5 (AAV8/5).

According to some embodiments, the delivery vectors are non-viral vectors. For example, the delivery vectors are synthetic gene delivery vehicles or vectors that are not related to a virus particle and that specifically deliver the gene material to the target cells or tissue. Examples of non-viral vectors include, but are not limited to, liposomes, peptides, nanoparticles, emulsions, or encapsulated two or more phase systems or other suitable preparation. Thus, according to some embodiments, the described invention provides a non-viral vector with nucleic acid that is loaded and contacted to a tissue or cell. By way of example, a liposome containing naked DNA encoding a human sCD59 protein having a modified GPI anchor that does not target a membrane, or a gene encoding a human sCD59 protein having no GPI anchor, is encapsulated in the liposome and the liposome is contacted to the tissue or cell such that the nucleic acid is effectively delivered to the tissue or cell.

According to some embodiments, the described invention provides a pharmaceutical composition. According to some embodiments, the pharmaceutical composition comprises a human sCD59 protein comprising a full-length nucleic acid of CD59 that was modified to remove the signal sequence for attachment of the GPI anchor at the nucleotides encoding amino acid asparagine at position 77. According to some embodiments, the nucleic acid sequence of human sCD59 protein is modified by point mutations, substitutions or deletions to obtain a nucleic acid sequence that encodes an amino acid sequence that has a modified amino acid sequence at the GPI anchor location, such that the protein is unable to attach to a membrane of a cell.

According to some embodiments, the described invention provides a pharmaceutical composition that comprises a CD59-encoding nucleic acid or a source of human sCD59 protein expression. In various embodiments, the CD59 protein includes a membrane-independent (i.e., soluble) CD59 protein. According to some embodiments, the pharmaceutical composition is compounded as an ophthalmologic formulation for administration to the eye. According to some embodiments, the pharmaceutical composition is compounded to enhance delivery to the fundus. According to some embodiments, the pharmaceutical composition is compounded to provide sustained release locally to the retina. According to some embodiments, the pharmaceutical composition is formulated to provide effective treatment of vessels and/or tissue involved in ocular diseases. According to some embodiments, the ocular disease is age-related macular degeneration (AMD). According to some embodiments, the AMD is wet or exudative AMD. According to some embodiments, the AMD is dry AMD or geographic atrophy (GA).

According to some embodiments, the pharmaceutical composition of the described invention is formulated sufficiently pure for administration to a human subject, e.g., to the eye of a human subject. According to some embodiments, the pharmaceutical composition includes one or more additional therapeutic agent(s). According to some embodiments, the additional therapeutic agent or agents are selected from the group consisting of growth factors, anti-inflammatory agents, vasopressor agents, including, but not limited to, nitric oxide and calcium channel blockers, collagenase inhibitors, steroids (e.g., prednisolone), matrix metalloproteinase inhibitors, ascorbates, angiotensin H, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid. According to some embodiments, the additional therapeutic agent or agents include, without limitation, anti-tumor, antiviral, antibacterial, anti-mycobacterial, anti-fungal, anti-proliferative or anti-apoptotic agents. Therapeutic agents that are included in the pharmaceutical composition of the described invention are well known in the art. See for example, Goodman & Gilman's The Pharmacological Basis of Therapeutics, 9th Ed., Hardman, et al., eds., McGraw-Hill, 1996, the contents of which are herein incorporated by reference herein.

According to some embodiments, the additional therapeutic agent or agents is a compound, composition, biologic or the like. According to some embodiments, the additional therapeutic agent or agents potentiate, stabilize, synergize or substitute for the ability of human sCD59 protein to protect cells from MAC deposition. According to some embodiments, the additional therapeutic agent or agents are provided at the same time as the pharmaceutical composition that comprises the human sCD59 protein. According to some embodiments, the additional therapeutic agent or agents are provided after the pharmaceutical composition that comprises the human sCD59 protein. According to some embodiments, the additional therapeutic agent or agents are provided before the pharmaceutical composition that comprises the human sCD59 protein. According to some embodiments, the additional therapeutic agent or agents are used to treat the same, a concurrent or a related symptom, condition or disease.

According to some embodiments, the pharmaceutical composition of the described invention comprises a pharmaceutically acceptable carrier. Pharmaceutical acceptable carriers include, but are not limited to, any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 provides various carriers used in formulating pharmaceutical compositions and known techniques for the preparation thereof. Some examples of materials which can serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants can also be present in the composition, according to the judgment of the formulator.

According to some embodiments, the described invention provides a method for treating a complement disorder (e.g., AMD). According to some embodiments, the method comprises contacting cells or tissue with a pharmaceutical composition comprising a source of human sCD59 protein. According to some embodiments, the pharmaceutical composition comprises a nucleotide sequence encoding human sCD59 operably linked to a promoter. According to some embodiments, the pharmaceutical composition comprises a human sCD59 expression construct. According to some embodiments, the pharmaceutical composition comprises a nucleotide sequence encoding human sCD59 operably linked to a promoter that is packaged into a delivery vector. According to some embodiments, the pharmaceutical composition comprises a human sCD59 expression construct that is packaged into a delivery vector.

According to some embodiments, the human sCD59 protein is administered as a recombinant protein.

Without being bound by theory, it is understood that plasma membranes of cells are normally protected from the effects of complement by cell-surface proteins, e.g., CD59, that specifically inhibit activation of the C5b-9 pore upon C9 complement protein binding to membrane C5b-8 (Holguin et al. 1989 J. Clin. Invest. 84: 7-17; Sims et al. 1989 J. Biol. Chem. 264: 19228-19235; Davies et al. 1989 J. Exp. Med. 170: 637-654; Rollins et al. 1990 J. Immunol. 144: 3478-3483; and Hamilton et al. 1990 Blood 76: 2572-2577). CD59 competes with C9 complement protein for binding to C8 complement protein in the C5b-8 complex, thereby decreasing or preventing the formation of the C5b-9 membrane attack complex. CD59 thus acts to reduce both cell activation and cell lysis by terminal complement MACs.

Theories have connected causation of disease such as age-related macular degeneration (AMD) and activation of the complement system and formation of MAC. Dinu (U.S. patent application number 2007/0196367 A1 published Aug. 23, 2007) proposes preventing debris formation by inhibiting complement as a therapeutic for AMD.

Diseases associated with uncontrolled complement activity include: bacterial infection such as with Haemophilus influenza, Streptococcus pnemoniae, Neisseria meningitidis; angiodema; renal disease for example atypical haemolytic uremic syndrome; paroxysmal nocturnal hemoglobinuria; systemic lupus erythematosus; central nervous system diseases including Alzheimer's disease, Huntington's disease and diseases of the retina including, but not limited to, age-related macular degeneration (AMD).

According to some embodiments, human soluble CD59 (sCD59) is effective to inhibit MAC formation. According to some embodiments, MAC formation is inhibited by delivering into a cell a vector containing a human sCD59-encoding nucleic acid.

According to some embodiments, human sCD59 is effective to treat AMD. According to some embodiments, AMD is treated by delivering into a cell a vector containing a human sCD59-encoding nucleic acid. According to some embodiments, human sCD59 According to some embodiments, human sCD59 is effective to prevent the onset of AMD. According to some embodiments, the onset of AMD is prevented by delivering into a cell a vector containing a human sCD59-encoding nucleic acid. According to some embodiments, human sCD59 is effective to prevent the progression of AMD. According to some embodiments, the progression of AMD is prevented by delivering into a cell a vector containing a human sCD59-encoding nucleic acid. According to some embodiments, human sCD59 is effective to reverse the progression of AMD. According to some embodiments, the progression of AMD is reversed by delivering into a cell a vector containing a human sCD59-encoding nucleic acid. According to some embodiments, the AMD is wet or exudative AMD. According to some embodiments, the AMD is dry AMD or geographic atrophy (GA).

According to some embodiments, human sCD59 is effective to attenuate choroidal neovascularization (CNV). According to some embodiments, human sCD59 is delivered by an approach using methods of gene therapy that is effective to attenuate choroidal neovascularization (CNV). According to some embodiments, human sCD59 is effective to reduce the extent of MAC deposition on CNV spots. According to some embodiments, the extent of MAC deposition on CNV spots is reduced by delivering into a cell a vector containing a human sCD59-encoding nucleic acid.

According to some embodiments, human sCD59 prevents lysis of retina cells. According to some embodiments, the lysis of retinal cells is prevented by delivering into a cell a vector containing a human sCD59-encoding nucleic acid.

According to some embodiments, human sCD59 is delivered by an adeno-associated virus (AAV) vector. According to some embodiments, the adeno-associated virus vector is AAV2. According to some embodiments, the adeno-associated virus is AAV5. According to some embodiments, the adeno-associated virus vector is AAV8. According to some embodiments, the AAV vector is a hybrid vector comprising ITR sequences from AAV2 and a capsid protein from AAV5 (AAV2/5). According to some embodiments, the AAV vector is a hybrid vector comprising ITR sequences from AAV2 and a capsid protein from AAV8 (AAV2/8). According to some embodiments, the AAV vector is a hybrid vector comprising ITR sequences from AAV5 and a capsid protein from AAV2 (AAV5/2). According to some embodiments, the AAV vector is a hybrid vector comprising ITR sequences from AAV5 and a capsid protein from AAV8 (AAV5/8). According to some embodiments, the AAV vector is a hybrid vector comprising ITR sequences from AAV8 and a capsid protein from AAV2 (AAV8/2). According to some embodiments, the AAV vector is a hybrid vector comprising ITR sequences from AAV8 and a capsid protein from AAV5 (AAV8/5).

According to some embodiments, the AAV vector is administered by injection. According to some embodiments, the injection is subretinal. According to some embodiments, the injection is intravitreal. According to some embodiments, the injection is a single injection. According to some embodiments, the injection is multiple injections.

Without being bound by theory, it is believed that contacting cells with a vector containing a human sCD59-encoding nucleic acid produces a subset of cells that are ‘factories’ for local production and secretion of sCD59, which may protect adjacent ocular cells including retinal pigment epithelium (RPE) cells and choroidal blood vessels.

Compositions and methods using nucleotide sequences encoding human sCD59 offer additional advantages over protein-based delivery methods. Peptides have limited half-lives in vivo and need to be re-administered on a regular basis. Current treatments for wet AMD include, for example, intraocular ranibizumab antibody injections every four to six weeks. This method of treatment exposes patients to complications and associated pathologies such as endophthalmitis. The incidence of endophthalmitis is relatively low (0.16% per dose) in the presence of a robust immune system. However, the rate of endophthalmitis increases substantially due to the cumulative effect of an attenuated complement system and serial injections over many years, such as for the treatment of chronic diseases such as AMD. Hence, frequent injection of complement inhibitors into the eyes of AMID patients is not desirable or effective. The pharmaceutical compositions and methods described herein limit the frequency of injection and therefore provide a more safe and effective treatment for subjects suffering from complement disorders such as AMD.

Viral vectors such as adenovirus vectors have been used to provide lifetime expression of transgenes in vivo in mice. AAV vectors, for example, have facilitated transgene expression in dogs for more than seven years. In humans, AAV has been found to have therapeutic transgene expression for over 3.7 years, the longest time periods studied. Adenovirus has been found to be an efficient vector for delivery of transgenes to ocular tissue and has been found to be safe in several ocular gene therapy trials. Adenovirus vectors engineered for long-term transgene expression and the technology for scaled production of such vectors are known in the art. AAV vectors have been shown to be safe for use in humans and are generally considered less immunogenic than adenovirus vectors.

According to some embodiments, delivery of human sCD59 to the eyes of AMD patients using an AAV vector is effective for long-term transgene expression.

According to some embodiments, the described invention provides a pharmaceutical composition for treating AMD comprising a vector carrying a nucleotide sequence encoding a recombinantly engineered human sCD59 protein operably linked to a promoter sequence causing expression of the protein in a cell, such that the nucleotide sequence carries at least one mutation conferring loss of a glycosylphosphatidylinositol (GPI) anchoring function, such that the protein is expressed as a recombinant membrane-independent (i.e., soluble) CD59 protein and is not membrane targeting. According to some embodiments, the pharmaceutical composition further comprises a pharmaceutically acceptable buffer. According to some embodiments, the AMD is wet or exudative AMD. According to some embodiments, the AMD is dry or GA.

According to some embodiments, the pharmaceutical composition is formulated sterile for ocular delivery. According to some embodiments, the pharmaceutical composition formulated for sterile ocular delivery is in a dose effective to treat AMD.

According to some embodiments, the pharmaceutical composition formulated for ocular delivery further includes at least one of a pharmaceutically acceptable buffer, a pharmaceutically acceptable salt and a pharmaceutically acceptable emollient suitable for delivery by at least one route selected from: intra-ocular injection, subconjunctival injection, subtenon injection, eye drop, and ointment.

According to some embodiments, the vector is at least one of: an engineered viral vector recombinantly linked to the nucleotide sequence encoding the sCD59 protein; and a synthetic gene delivery vector for delivery of the nucleotide sequence encoding human sCD59. According to some embodiments, the viral vector is selected from the group consisting of adenovirus, adeno-associated virus, a herpesvirus, a poxvirus, and a lentivirus. According to some embodiments, the synthetic gene delivery vector is selected from the group consisting of a liposome, a lipid/polycation (LPD), a peptide, a nanoparticle, a gold particle, and a polymer.

According to some embodiments, the pharmaceutical composition further includes a peptide for overall delivery (POD), the pharmaceutical composition operably linked to the compound to obtain a conjugated compound, such that the POD includes a protein transduction domain (PTD). For example, the POD composition is one shown in Kumar-Singh et al. PCT/US2008/010179 filed Aug. 28, 2008 or Kumar-Singh et al. U.S. publication 2010/0209447 published Aug. 19, 2010, each of which is incorporated herein by reference in its entirety.

According to some embodiments, the pharmaceutical composition comprises a dose of viral vector particles administered to an affected eye. According to some embodiments, the dose of viral particles ranges from about 1×10⁷ to about 1×10⁹. According to some embodiments, the dose of viral particles ranges from about 1×10⁸ to about 1×10¹⁰. According to some embodiments, the dose of viral particles ranges from about 1×10⁹ to about 1×10¹¹. According to some embodiments, the dose of viral particles ranges from about 1×10¹¹ to about 1×10¹². According to some embodiments, the dose of viral particles ranges from about 1×10¹¹ to about 1×10¹³. According to some embodiments, the pharmaceutical composition further includes at least one therapeutic agent selected from the group consisting of an anti-inflammatory, an anti-tumor, an antiviral, an antibacterial, an anti-mycobacterial, an anti-fungal, an anti-proliferative and an anti-apoptotic. According to some embodiments, the dose of viral particles ranges from about 1×10¹⁰ DNAse-resistant particles (DRP) to about 1×10¹² DRP. According to some embodiments, the dose of viral particles is about 3.56×10¹⁰ DRP. According to some embodiments, the dose of viral particles is about 1.071×10¹¹ DRP. According to some embodiments, the dose of viral particles is about 3.56×10¹¹ DRP. According to some embodiments, the dose of viral particles is about 1.07×10¹² DRP.

According to some embodiments, the pharmaceutical composition comprises a promoter sequence. According to some embodiments, the promoter sequence is a ubiquitous promoter for general for expression in a mammalian cell. According to some embodiments, the promoter is a promoter from a gene encoding actin, polyhedron, or hydroxyl-methylglutaryl CoA reductase (HMGCR). Such promoters include, but are not limited to, a chicken beta-actin promoter or a human beta-actin promoter. According to some embodiments, the promoter sequence is a tissue specific promoter for expression in a specific cell-type. Specific cell-type promoters include, but are not limited to, a rhodopsin promoter or tissue specific promoter for the eye or liver.

According to some embodiments, the described invention provides a method for formulating a composition for treating age-related macular degeneration (AMD) in a subject, the method comprising engineering a vector to deliver and express a human sCD59 nucleotide sequence encoding an amino acid sequence corresponding to human sCD59, such that the nucleotide sequence includes a mutation encoding for amino acids of a glycosyl phosphatidyl inositol (GPI) anchoring domain of the protein, such that the resulting vector encodes an engineered recombinant membrane-independent (i.e., soluble) CD59 (sCD59) protein, and the vector is a viral vector or a synthetic gene delivery vector; and, contacting at least one ocular tissue of the subject with the composition, such that the cells of the tissue express and secrete the CD59 locally, thereby treating the subject for AMD.

According to some embodiments, the viral vector is derived from a genetically engineered genome of at least one virus selected from the group consisting of an adenovirus, an adeno-associated virus, a herpesvirus, and a lentivirus.

According to some embodiments, the synthetic gene delivery vector is selected from the group consisting of a liposome, a lipid/polycation (LPD), a peptide, a nanoparticle, a gold particle, and a polymer.

According to some embodiments, contacting at least one ocular tissue of the subject further includes injecting by a route selected from the group consisting of intravitreal, subretinal, subconjunctival, subtenon; subcutaneous and intravenous. According to some embodiments, the tissues contacted by the pharmaceutical composition comprises at least one tissue selected from the group consisting of retinal pigment epithelium, retina, choroid, sclera, Bruch's membrane and choroidal blood vessels.

According to some embodiment, the described invention provides a method of regulating complement activity or treating a complement activity disorder in a subject, the method comprising contacting an affected tissue or organ of the subject at risk for or suffering from the complement activity disorder with a composition including a vector carrying a nucleotide sequence encoding a recombinantly engineered human sCD59 protein operably linked to a promoter sequence causing expression of the protein in a cell, such that the protein includes at least one mutation resulting in loss of function of glycosylphosphatidylinositol (GPI) anchoring domain, such that the protein is recombinant membrane-independent (i.e., soluble) CD59 (sCD59) and is not membrane targeting; and, observing a physiological indicium of the complement activity disorder after contacting, in comparison to an abnormal amount of the physiological indicium observed prior to contacting, such that a decrease after contacting compared prior to contacting is a positive indication that the affected tissue or organ is treated.

According to some embodiments, the affected tissue is selected from the group consisting of epithelial tissue, endothelial tissue and vascular tissue. According to some embodiments, the affected organ is selected from the group consisting of eye, heart, kidney, lung, liver, pancreas and vascular system. According to some embodiments, the subject is a tissue or organ donor or recipient. According to some embodiments, the subject is an immunocompromised patient that is an organ recipient.

According to some embodiments, the described method comprises treating a disorder selected from the group consisting of age-related macular degeneration (AMD), bacterial infection, toxic shock syndrome (TSS), atypical hemolytic uremic syndrome, membranoproliferative glomerulonephritis, dense deposit disease, peroximal nocturnal hemoglobinurea, systemic lupus erythromatosis, atherosclerosis and the like. According to some embodiments, the AMD is wet or exudative AMD. According to some embodiments, the AMD is dry AMD or geographic atrophy (GA). According to some embodiments, the disorder is AMD. According to some embodiments, the AMD is dry AMD (GA). According to some embodiments, the observing further includes measuring the indication selected from the group consisting of visual acuity, visual aberrations and amount of MAC deposition.

According to some embodiments, the described invention provides a method of treating a complement disorder comprising contacting a tissue or a cell with a pharmaceutical composition. According to some embodiments, the method comprises administering a therapeutically effective amount of a pharmaceutical composition having as an active agent a nucleic acid encoding a human sCD59 protein or a source of expression of a human sCD59 protein, to a subject in need thereof, in such amounts and for such time as is necessary to achieve the desired result. According to some embodiments, the method comprises treating AMD by contacting an ocular tissue or cell with huma sCD59 protein or a vector encoding human sCD59 protein.

According to some embodiments, the pharmaceutical composition is administered using any amount and any route of administration effective for treating AMD or other complement-related diseases and conditions. Thus, the expression “amount effective for treating AMD”, as used herein, refers to a sufficient amount of the pharmaceutical composition to beneficially prevent or ameliorate the symptoms of AMD.

The exact dosage of the pharmaceutical composition may be chosen by the individual physician in view of the patient to be treated. Dosage and administration are adjusted to provide sufficient levels of the active agent(s) or to maintain the desired effect. Additional factors which may be taken into account include the severity of the disease state, e.g., intermediate or advanced stage of AMD; age, weight and gender of the patient; diet, time and frequency of administration; route of administration; drug combinations; reaction sensitivities; and tolerance/response to therapy. Long-acting pharmaceutical compositions may be administered one time, hourly, twice hourly, every 3 to four hours, once daily, twice daily, every 3 to 4 days, every week, or once every two weeks depending on half-life and clearance rate of the particular composition.

The active agents of the described invention can be formulated in dosage unit form for ease of administration and uniformity of dosage. It will be understood, however, that the total daily usage of the compositions of the present invention will be decided by the attending physician within the scope of sound medical judgment. For any active agent, the therapeutically effective dose can be estimated initially either in cell culture assays or in animal models, as provided herein, usually mice, but also potentially from rats, rabbits, dogs, or pigs. Such information can then be used to determine useful doses and routes of administration for humans. Pharmaceutical compositions which exhibit large therapeutic indices are preferred. The data obtained from cell culture assays and animal studies are used in formulating a range of dosage for human use.

A therapeutically effective dose refers to that amount of active agent that ameliorates the symptoms or condition or prevents progression of AMD. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50.

The daily dosage of a pharmaceutical composition may be varied over a wide range, such as from 0.001 to 100 mg per adult human per day. For ocular administration, pharmaceutical compositions may be provided in the form of a solution containing 0.001, 0.01, 0.05, 0.1, 0.5, 1.0, 2.5, 5.0, 10.0, 15.0, 25.0, 50.0, 100.0, 250.0, or 500.0 micrograms (μg) of the active ingredient for the symptomatic adjustment of the dosage to the patient to be treated.

A unit dose typically contains from about 0.001 micrograms to about 500 micrograms of the active ingredient, from about 0.1 micrograms to about 100 micrograms of active ingredient, or from about 1.0 micrograms to about 10 micrograms of active ingredient. An effective amount of a drug may be supplied at a dosage level of from about 0.0001 mg/kg to about 25 mg/kg of body weight per day. For example, the range maybe from about 0.001 to 10 mg/kg of body weight per day, or from about 0.001 mg/kg to 1 mg/kg of body weight per day. Pharmaceutical compositions may be administered on a regimen of, for example, one to four or more times per day. A unit dose may be divided and administered, for example, in two or more divided doses.

According to some embodiments, a source of expression of a human sCD59 protein is administered as a dose of a viral vector or a nucleic acid vector, such that the dose contains at least about 50, 100, 500, 1000, or at least about 5000 particles per cell to be treated. Cell number can be calculated from retinal area in need of treatment by methods known to one of skill in the art. According to some embodiments, the dose ranges from about 1×10¹⁰ DNAse-resistant particles (DRP) to about 1×10¹² DRP. According to some embodiments, the dose is about 3.56×10¹⁰ DRP. According to some embodiments, the dose is about 1.071×10¹¹ DRP. According to some embodiments, the dose is about 3.56×10¹¹ DRP. According to some embodiments, the dose is about 1.07×10¹² DRP.

According to some embodiments, the source of expression of human sCD59 protein is administered by ocular injections. Ocular injections include, but are not limited to, intra-ocular injection into the aqueous or the vitreous humor, or injection into the external layers of the eye, such as via subconjunctival injection or subtenon injection.

Injectable preparations, such as sterile injectable aqueous or oleaginous suspensions, may be formulated according to the known art using suitable dispersing or wetting agents and suspending agents. According to some embodiments, the injectable preparation is a sterile injectable preparation. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. Any bland, fixed oil can be employed including, but not limited to, synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

According to some embodiments, the sterile injectable preparation includes excipients. Such excipients include, without limitation, suspending agents (e.g., sodium carboxymethylcellulose, methylcellulose, hydroxy-propylmethylcellulose, sodium alginate, polyvinylpyrrolidone, gum tragacanth, and gum acacia), dispersing or wetting agents including, a naturally-occurring phosphatide (e.g., lecithin), or condensation products of an alkylene oxide with fatty acids (e.g., polyoxyethylene stearate), or condensation products of ethylene oxide with long chain aliphatic alcohols (e.g., heptadecaethyl-eneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate).

The sterile injectable preparation may also be a sterile injectable solution or suspension in a nontoxic parenterally-acceptable or diluent or solvent, for example, as a solution in 1, 3-butanediol. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it does not rapidly settle out. In everyday life, the most common suspensions are those of solids in liquid water. Among the acceptable vehicles and solvents that may be employed are water, Ringer's solution, and isotonic sodium chloride solution. In addition, sterile, fixed oils are conventionally employed as a solvent or suspending medium. For parenteral application, particularly suitable vehicles consist of solutions, preferably oily or aqueous solutions, as well as suspensions, emulsions, or implants. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension also may contain suitable stabilizers or agents, which increase the solubility of the compounds to allow for the preparation of highly concentrated solutions. Alternatively, the active compounds may be in powder form for constitution with a suitable vehicle, e.g., sterile pyrogen-free water, before use.

According to some embodiments, the described invention provides liquid dosage forms for ocular injection. Such liquid dosage forms include, but are not limited to, pharmaceutically acceptable emulsions, microemulsions, solutions, suspensions, syrups and elixirs. In addition to the active agent(s), the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In addition to inert diluents, ocular-delivered pharmaceutical compositions can also include adjuvants such as wetting agents and emulsifying and suspending agents.

According to some embodiments, the pharmaceutical composition of the described invention may be in the form of a sterile injectable aqueous or oleaginous suspension. Injectable preparations, such as sterile injectable aqueous or oleaginous suspensions, may be formulated according to the known art using suitable dispersing or wetting agents and suspending agents.

According to some embodiments, the described invention comprises ophthalmological devices, surgical devices, audiological devices or products which contain disclosed compositions (e.g., gauze bandages or strips), and methods of making or using such devices or products. These devices may be coated with, impregnated with, bonded to, or otherwise treated with the pharmaceutical composition described herein.

According to some embodiments, the described invention provides administering the pharmaceutical composition to a subject. According to some embodiment, the step of administering comprises oral administration, topical administration or parenteral administration. According to some embodiments, parenteral administration is selected from the group consisting of intravitreal injection and subretinal injection.

According to some embodiments the administering step comprises administering the pharmaceutical composition as a single dose or as multiple doses. According to some embodiments, the administering step comprises administering the pharmaceutical composition as a single dose. According to some embodiments, the single dose is administered to the eye of a subject in need thereof. According to some embodiments, the subject in need thereof is suffering from AMD. According to some embodiments, the subject in need thereof is suffering from wet or exudative AMD. According to some embodiments, the subject in need thereof is suffering from GA.

According to some embodiments, the composition is administered in a pharmaceutically acceptable solution, which may routinely contain pharmaceutically acceptable concentrations of salt, buffering agents, preservatives, compatible carriers, adjuvants, and optionally other therapeutic agents.

According to some embodiments, the pharmaceutical composition is an aqueous suspension or emulsion in admixture with excipients suitable for the manufacture of aqueous suspensions and emulsions. Such excipients include, but are not limited to, suspending agents, such as sodium carboxymethylcellulose, methylcellulose, hydroxypropylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

Solutions or suspensions used for parenteral, intradermal, subcutaneous, intrathecal, or topical application may include, but are not limited to, a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation may be enclosed in ampoules, disposable syringes or multiple dose vials made of glass or plastic. Administered intravenously, particular carriers are physiological saline or phosphate buffered saline (PBS).

The injectable formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

The pharmaceutical composition of the described invention may further include conventional excipients, i.e., pharmaceutically acceptable organic or inorganic carrier substances suitable for parenteral application which do not deleteriously react with the active compounds. Suitable pharmaceutically acceptable carriers include, but are not limited to, water, salt solutions, alcohol, vegetable oils, polyethylene glycols, gelatin, lactose, amylose, magnesium stearate, talc, silicic acid, viscous paraffin, perfume oil; fatty acid monoglycerides and diglycerides, petroethral fatty acid esters, hydroxymethylcellulose, polyvinylpyrrolidone, etc.

The pharmaceutical composition of the described invention may be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, suitable vehicles include solutions, such as oily or aqueous solutions, as well as suspensions, emulsions, or implants. Aqueous suspensions may contain substances which increase the viscosity of the suspension and include, for example, but not limited to, sodium carboxymethyl cellulose, sorbitol and/or dextran. Optionally, the suspension also may contain stabilizers. These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

According to some embodiments, the pharmaceutical composition of the described invention comprises a therapeutically effective amount of human sCD59 and optionally other therapeutic agents included in a pharmaceutically-acceptable carrier. According to some embodiments, the components of the pharmaceutical composition also are capable of being commingled in a manner such that there is no interaction which would substantially impair the desired pharmaceutical efficacy.

According to some embodiments, the pharmaceutical composition of the described invention includes a pharmaceutically acceptable salt. Pharmaceutically acceptable salts are those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which can independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “an”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g., amounts, temperatures, etc.) but some experimental errors and deviations should be accounted for. Unless indicated otherwise, parts are by weight, molecular weight is weight average molecular weight, temperature is in degrees Centigrade, and pressure is at or near atmospheric.

### Example 1: Adenovirus Vector Constructs Expressing CD59

Adenovirus vector constructs expressing human soluble CD59 (sCD59) were prepared as described in U.S. Pat. Nos. 8,324,182 and 10,351,617. Briefly, human CD59 cDNA was obtained from the American Type Tissue Culture Collection (ATCC, Manassas, Va.). Human CD59 lacking the sequence coding for the C terminal 26 amino acids, which includes a signal sequence for attachment of the glycosylphosphatidylinositol (GPI) anchor was PCR amplified using a forward primer containing an XhoI site (5′ccccctcgagtggacaatcacaatggg3′; SEQ ID NO: 1) and a reverse primer with an EcoRV site (5′taaggagatatcttaattttcaagctgttcgtta3′; SEQ ID NO: 2). The reverse primer introduced a stop codon following Asparagine 77 resulting in a sequence that encodes a soluble form of human CD59 (sCD59) (MGIQGGSVLFGLLLVLAVFCHSGHSLQCYNCPNPTADCKTAVNCSSDFDACLITKAGLQ VYNKCWKFEHCNFNDVTTRLRENELTYYCCKKDLCNFNEQLEN; SEQ ID NO: 3). The PCR product was gel purified and XhoI/EcoRV digested. The XhoI/EcoRV digested PCR product was cloned into XhoI/EcoRV digested pShCAG and the resulting plasmid pShCAGsCD59 was used to produce adenovirus AAVCAGsCD59 using protocols known in the art (e.g., Klein et al. 2007 Ophthalmology 114: 253-262, and van Leeuwen et al. 2003 Eur. J. Epidemiol. 18: 845-854). Thus, the GPI signal was removed by recombinant methods to obtain a construct that expresses a soluble, secreted version of human CD59.

### Example 2: Phase 1/2a, Open-Label, Single-Site, Dose-Escalating, Safety and Tolerability Study of a Single Intravitreal Injection of AAVCAGsCD59 in Patients with Advanced Non-Exudative (Dry) Age-Related Macular Degeneration with Geographic Atrophy

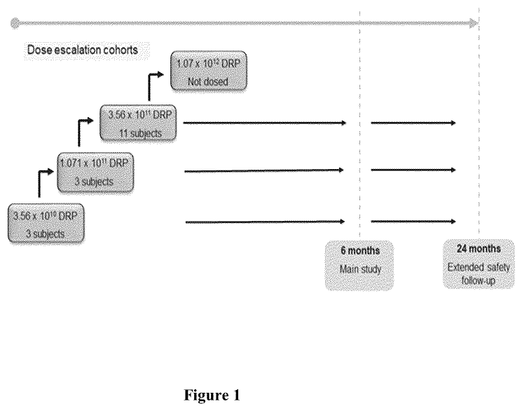

In this study, an open-label, non-randomized, Phase I, dose-escalation study was conducted to establish the safety of a single intravitreal injection of gene therapy vector AAVCAGsCD59 (adeno-associated viral vector serotype 2) that expresses soluble CD59 (sCD59), an inhibitor of the membrane attack complex (MAC), for the treatment of patients with advanced dry age-related macular degeneration (AMD) with geographic atrophy (GA). The study schema is shown in FIG. 1. The planned total sample size was approximately 26 participants. Seventeen participants were ultimately enrolled. The objectives and endpoints of this study are listed in Table 1. The number of participants (planned and analyzed) are listed in Table 2.

TABLE 1
Objectives and Endpoints

| Objectives | Endpoints |
|---|---|
| Primary | |
| To evaluate the safety and tolerability of a | Ocular (study eye and fellow eye separately) |
| single uniocular intravitreal injection of | and non-ocular treatment-emergent adverse |
| AAVCAGsCD59 in eyes with advanced dry | events (TEAEs) including adverse events |
| age-related macular degeneration (AMD) | (AEs), serious AEs (SAEs), AEs leading to |
| with GA | termination of study participation, AEs by |
| | severity (Common Terminology Criteria for |
| | Adverse Events [CTCAE] version 4), AEs |
| | by causality to AAVCAGsCD59, and death |
| | Clinical laboratory data of the worst |
| | treatment-emergent National Cancer |
| | Institute (NCI)-CTCAE grade by dose level |
| | and overall, and selected laboratory |
| | parameters by visit and cohort |
| | Change from baseline through Week 26 in |
| | vital sign parameters |
| | Titers of neutralizing serum anti-AAV2 |
| | antibodies, titers of serum anti-sCD59 |
| | antibodies, and serum AAV2CAGsCD59 |
| | vector distribution |
| | Intraocular pressure (IOP) absolute values |
| | and changes from baseline by |
| | visit on the study eye and the fellow |
| | eye separately |
| | Abnormality of slit lamp biomicroscopy and |
| | indirect/dilated ophthalmoscopy by selected |
| | visit |
| Secondary | |
| To evaluate the change in area of GA in | GA lesion area in square root transformed |
| eyes with dry AMD | value and its change from baseline at |
| To evaluate the rate of growth of GA in | selected visits |
| eyes with dry AMD | GA lesion area absolute value and percent |
| Incidence of conversion of dry AMD to wet | change from baseline at selected visits |
| AMD | Number and percentage of participants with |
| Evaluation of drusen volume | incidence of conversion of dry AMD to wet |
| Prevention of loss of 15 or more letters on | AMD by selected visits |
| an Early Treatment Diabetic Retinopathy | Drusen volume absolute value and absolute |
| Study (ETDRS) chart | change from baseline at selected visits |
| | Distance best corrected visual acuity |
| | (BCVA) (letters) absolute value and |
| | absolute change from baseline at visits |
| | Number and percentage of participants with |
| | a loss of ≥10, ≥15, ≥20, and ≥30 letters at |
| | least once in distance BCVA from baseline |
| | over time |
| | Number and percentage of participants with |
| | a loss of≥10, ≥15, ≥20, and ≥30 letters in |
| | distance BCVA from baseline by visit |
| | Number and percentage of participants who |
| | maintain or gain ≥0, ≥5, ≥10, and ≥15 letters |
| | at least once in distance BCVA from |
| | baseline over time |
| | Number and percentage of participants who |
| | maintain or gain ≥0, ≥5, ≥10, and ≥15 letters |
| | in distance BCVA from baseline by visit |
| Exploratory | |
| To evaluate protein expression of sCD59 in | sCD59 protein levels in the aqueous humor |
| the aqueous humor | |

TABLE 2
Number of Participants (Planned and Analyzed)
Study Completion/Withdrawal Information; Safety Analysis Set (Study MDG1001)

| | Cohort 1 | Cohort 2 | Cohort 3 | | Total | |
|---|---|---|---|---|---|---|
| Analysis Set: Safety | 3 | 3 | 11 | | 17 | |
| Analysis Set | | | | | | |
| Completed Study | 3 (100.0%) | 3 (100.0%) | 10 | (90.9%) | 16 | (94.1%) |
| Discontinued Study | 0 | 0 | 1 | (9.1%) | 1 | (5.9%) |
| Patients who completed | 3 (100.0%) | 3 (100.0%) | 11 | (100.0%) | 17 | (100.0%) |
| the study (at Week 26) | | | | | | |
| Patients who completed | 3 (100.0%) | 3 (100.0%) | 10 | (90.9%) | 16 | (94.1%) |
| the long- term follow- | | | | | | |
| up (2 years) | | | | | | |
| Reason for Study | | | | | | |
| Discontinuation | | | | | | |
| Death | 0 | 0 | 1 | (9.1%) | 1 | (5.9%) |

Cohort 1 = 3.56 × 10¹⁰ DNAse-resistant particles (DRP)
Cohort 2 = 1.071 × 10¹¹ DNAse-resistant particles (DRP)
Cohort 3 = 3.56 × 10¹¹ DNAse-resistant particles (DRP)

### Study Population; Inclusion/Exclusion Criteria

The study population consisted of adult men or women, 50 years of age or older, with advanced dry AMD with GA in the study eye. Participants had a BCVA Snellen equivalent of 20/200 or worse in the study eye for the first 3 participants, and then a BCVA Snellen equivalent of 20/80 or worse in the study eye after the first 3 participants. Total GA lesion size was 5 mm² (2 disc areas (DA)) to 20 mm² (8 DA) in the study eye, and BCVA of 20/800 or better in the fellow eye.

Participants who had GA secondary to non-AMD etiologies, prior or active choroidal neovascularization (CNV) in the study eye, active or uncontrolled glaucoma, had, or were likely candidates for, intraocular surgery in the study eye, or had acute or chronic infection in the study eye were excluded.

### Disposition of Participants

Seventeen (17) participants (100%) completed the study at Week 26 and 16 participants (94.1%) completed the 2 year long-term follow-up study (Table 2). One (1) participant (5.9%) discontinued from the study prematurely due to death not related to study intervention (Table 2).

### Demographic and Other Baseline Characteristics

A higher proportion of participants were female (64.7%), and all participants were white. The median age was 81 years (range 69 to 95 years) (Table 3). The mean body mass index (BMI) was 28.5 kg/m² (range 20 to 39 kg/m²). The mean BMI was higher for Cohort 1 than in Cohorts 2 or 3 (32.9 kg/m² vs 27.7 kg/m² and 27.5 kg/m², respectively.

TABLE 3
Summary of Demographic and Baseline Characteristics

| | Cohort 1 | | Cohort 2 | | Cohort 3 | | Total | |
|---|---|---|---|---|---|---|---|---|
| Analysis set: Safety | 3 | | 3 | | 11 | | 17 | |
| Analysis set | | | | | | | | |
| Age, years | | | | | | | | |
| N | 3 | | 3 | | 11 | | 17 | |
| Mean (SD) | 81.0 | (2.65) | 85.7 | (8.33) | 79.7 | (6.84) | 81.0 | (6.63) |
| Median | 80.0 | | 83.0 | | 81.0 | | 81.0 | |
| Range | (79; 84) | | (79; 95) | | (69; 93) | | (69; 95) | |
| <65 years | 0 | | 0 | | 0 | | 0 | |
| >=65 years | 3 | (100.0%) | 3 | (100.0%) | 11 | (100.0%) | 17 | (100.0%) |
| Sex | | | | | | | | |
| N | 3 | | 3 | | 11 | | 17 | |
| Female | 0 | | 3 | (100.0%) | 8 | (72.7%) | 11 | (64.7%) |
| Male | 3 | (100.0%) | 0 | | 3 | (27.3%) | 6 | (35.3%) |
| Race | | | | | | | | |
| N | 3 | | 3 | | 11 | | 17 | |
| American Indian or | 0 | | 0 | | 0 | | 0 | |
| Alaska Native | | | | | | | | |
| Asian | 0 | | 0 | | 0 | | 0 | |
| Black or African | 0 | | 0 | | 0 | | 0 | |
| American | | | | | | | | |
| Native Hawaiian or | 0 | | 0 | | 0 | | 0 | |
| other Pacific Islander | | | | | | | | |
| White | 3 | (100.0%) | 3 | (100.0%) | 11 | (100.0%) | 17 | (100.0%) |
| Not Reported | 0 | | 0 | | 0 | | 0 | |
| Ethnicity | | | | | | | | |
| N | 3 | | 3 | | 11 | | 17 | |
| Hispanic or Latino | 0 | | 0 | | 0 | | 0 | |
| Not Hispanic or Latino | 3 | (100.0%) | 3 | (100.0%) | 11 | (100.0%) | 17 | (100.0%) |
| Not Reported | 0 | | 0 | | 0 | | 0 | |
| Weight, kg | | | | | | | | |
| N | 3 | | 3 | | 11 | | 17 | |
| Mean (SD) | 105.75 | (10.311) | 71.25 | (22.198) | 70.69 | (10.898) | 76.98 | (18.375) |
| Median | 103.50 | | 60.75 | | 69.75 | | 72.00 | |
| Range | (96.8; 117.0) | | (56.3; 96.8) | | (45.0; 86.4) | | (45.0; 117.0) | |
| Height, cm | | | | | | | | |
| N | 3 | | 3 | | 11 | | 17 | |
| Mean (SD) | 180.34 | (9.158) | 160.02 | (8.799) | 160.25 | (6.446) | 163.76 | (10.429) |
| Median | 177.80 | | 165.10 | | 158.75 | | 163.83 | |
| Range | (172.7; 190.5) | | (149.9; 165.1) | | (149.9; 170.2) | | (149.9; 190.5) | |
| Body mass index, kg/m2 | | | | | | | | |
| N | 3 | | 3 | | 11 | | 17 | |
| Mean (SD) | 32.9 | (6.28) | 27.7 | (7.45) | 27.5 | (3.84) | 28.5 | (5.05) |
| Median | 32.7 | | 27.1 | | 27.3 | | 27.3 | |
| Range | (27; 39) | | (21; 35) | | (20; 32) | | (20; 39) | |
| Smoking History | | | | | | | | |
| N | 3 | | 3 | | 11 | | 17 | |
| Current | 0 | | 0 | | 1 | (9.1%) | 1 | (5.9%) |
| Former | 2 | (66.7%) | 1 | (33.3%) | 4 | (36.4%) | 7 | (41.2%) |
| Never | 1 | (33.3%) | 2 | (66.7%) | 6 | (54.5%) | 9 | (52.9%) |
| Number of cigarette | | | | | | | | |
| packs smoked per day | | | | | | | | |
| N | 2 | | 1 | | 5 | | 8 | |
| Mean (SD) | 1.10 | (1.273) | 1.50 | (—) | 0.58 | (0.407) | 0.82 | (0.675) |

| | | | | |
|---|---|---|---|---|
| Median | 1.10 | 1.50 | 0.50 | 0.75 |
| Range | (0.2; 2.0) | (1.5; 1.5) | (0.1; 1.0) | (0.1; 2.0) |

KEY:
DRP = DNase-Resistant Particles,
SD = standard deviation
Cohort 1 = 3.56 × 10¹⁰ DRP
Cohort 2 = 1.071 × 10¹¹ DRP
Cohort 3 = 3.56 × 10¹¹ DRP

### Ocular Baseline Characteristics

### Study Eye

All participants had advanced dry AMD with GA with a mean duration from first diagnosis of AMD of 12.13 years (range 2.25 to 42.67 years). Mean visual acuity was 37.26 ETDRS letters overall. Per protocol, participants in Cohort 1 had lower mean visual acuity than those in either Cohorts 2 or 3 (18.67 ETDRS letters [Cohort 1], 50.50 ETDRS letters [Cohort 2], and 38.73 ETDRS letters [Cohort 3]). Mean IOP was similar across all cohorts with a mean IOP of 13.82 mm Hg. Table 4 summarizes the ocular baseline characteristics for the study eye.

TABLE 4
Summary of Study Eye Ocular Baseline Characteristics

| | Cohort 1 | | Cohort 2 | | Cohort 3 | | Total | |
|---|---|---|---|---|---|---|---|---|
| Analysis set: Safety | 3 | | 3 | | 11 | | 17 | |
| analysis set | | | | | | | | |
| Study Eye | | | | | | | | |
| N | 3 | | 3 | | 11 | | 17 | |
| Left | 0 | | 0 | | 8 | (72.7%) | 8 | (47.1%) |
| Right | 3 | (100.0%) | 3 | (100.0%) | 3 | (27.3%) | 9 | (52.9%) |
| History of Advanced Dry | | | | | | | | |
| AMD with Geographic | | | | | | | | |
| Atrophy | | | | | | | | |
| N | 3 | | 3 | | 11 | | 17 | |
| Yes-Ongoing | 3 | (100.0%) | 3 | (100.0%) | 11 | (100.0%) | 17 | (100.0%) |
| Duration of AMD (month) | | | | | | | | |
| N | 3 | | 3 | | 11 | | 17 | |
| Mean (SD) | 95.00 | (79.228) | 220.33 | (198.031) | 139.00 | (163.686) | 145.59 | (154.886) |
| Median | 76.00 | | 125.00 | | 70.00 | | 88.00 | |
| Range | (27.0; 182.0) | | (88.0; 448.0) | | (32.0; 512.0) | | (27.0; 512.0) | |
| Distance BCVA (letters) | | | | | | | | |
| N | 3 | | 3 | | 11 | | 17 | |
| Mean (SD) | 18.67 | (6.658) | 50.50 | (2.500) | 38.73 | (11.648) | 37.26 | (13.794) |
| Median | 17.00 | | 50.50 | | 36.00 | | 36.00 | |
| Range | (13.0; 26.0) | | (48.0; 53.0) | | (22.5; 56.5) | | (13.0; 56.5) | |
| IOP (mm Hg) | | | | | | | | |
| N | 3 | | 3 | | 11 | | 17 | |
| Mean (SD) | 13.33 | (2.930) | 13.00 | (1.803) | 14.18 | (3.509) | 13.82 | (3.072) |

| | | | | |
|---|---|---|---|---|
| Median | 14.50 | 12.50 | 14.00 | 14.00 |
| Range | (10.0; 15.5) | (11.5; 15.0) | (9.5; 21.5) | (9.5; 21.5) |

Key:
AMD = age-related macular degeneration,
BCVA = best corrected visual acuity,
DRP = DNase-Resistant Particles,
SD = standard deviation
Cohort 1 = 3.56 × 10¹⁰ DRP
Cohort 2 = 1.071 × 10¹¹ DRP
Cohort 3 = 3.56 × 10¹¹ DRP

### Fellow Eye

Fifteen (15) out of 17 eyes had a history of advanced dry AMD with GA in the fellow eye. Mean duration of AMD from first diagnosis was 13.02 years (range 2.25 to 42.67 years). Mean BCVA in the fellow eye was 44.00, 59.17, and 59.59 ETDRS letters in Cohorts 1, 2, and 3, respectively, with an overall mean BCVA of 56.76 ETDRS letters. Intraocular pressure (IOP) was within normal limits (mean 13.88 mm Hg) and similar across all cohorts

### GA Lesions

### Study Eye

Mean baseline GA lesion size in the study eye was 11.12 mm² and was similar across the 3 cohorts. All participants had foveal-involving GA lesions. Fundus autofluorescence (FAF) patterns at the junction zone included banded (11 participants [64.7%]), diffuse (4 participants [23.5%]), and focal (2 participants [11.8%]). No CNV was noted in the study eye by fluorescein angiography (FA).

TABLE 5
Summary of Study Eye Imaging Baseline Characteristics

| | Cohort 1 | | Cohort 2 | | Cohort 3 | | Total | |
|---|---|---|---|---|---|---|---|---|
| Analysis set: Safety | 3 | | 3 | | 11 | | 17 | |
| analysis set | | | | | | | | |
| FAF Geographic | | | | | | | | |
| Atrophy | | | | | | | | |
| N | 3 | | 3 | | 11 | | 17 | |
| Yes | 3 | (100.0%) | 3 | (100.0%) | 11 | (100.0%) | 17 | (100.0%) |
| FAF Geographic | | | | | | | | |
| Atrophy Location | | | | | | | | |
| N | 3 | | 3 | | 11 | | 17 | |
| Foveal | 3 | (100.0%) | 3 | (100.0%) | 11 | (100.0%) | 17 | (100.0%) |
| FAF Geographic | | | | | | | | |
| Atrophy Area (mm2) | | | | | | | | |
| N | 3 | | 3 | | 11 | | 17 | |
| Mean (SD) | 11.73 | (3.219) | 11.47 | (2.075) | 10.87 | (5.004) | 11.12 | (4.197) |
| Median | 12.64 | | 12.48 | | 8.29 | | 10.22 | |
| Range | (8.2; 14.4) | | (9.1; 12.8) | | (5.5; 19.5) | | (5.5; 19.5) | |
| FAF at Junctional | | | | | | | | |
| Zone Pattern | | | | | | | | |
| N | 3 | | 3 | | 11 | | 17 | |
| Banded | 2 | (66.7%) | 2 | (66.7%) | 7 | (63.6%) | 11 | (64.7%) |
| Diffuse | 1 | (33.3%) | 1 | (33.3%) | 2 | (18.2%) | 4 | (23.5%) |
| Focal | 0 | | 0 | | 2 | (18.2%) | 2 | (11.8%) |
| FA CNV | | | | | | | | |
| N | 3 | | 3 | | 11 | | 17 | |
| No | 3 | (100.0%) | 3 | (100.0%) | 11 | (100.0%) | 17 | (100.0%) |

KEY:
DRP = DNase-Resistant Particles,
FA CNV = fluorescein angiography choroidal neovascularization,
FAF = fundus autofluoreschort
1 = ho
Cohort 1 = 3.56 × 10¹⁰ DNase-Resistant Particles (DRP)
Cohort 2 = 1.071 × 10¹¹ DNase-Resistant Particles (DRP)
Cohort 3 = 3.56 × 10¹¹ DNase-Resistant Particles (DRP)

### Fellow Eye

Fourteen (14) participants (82.4%) had GA in the fellow eye. Thirteen (13) (92.9%) of these participants had lesions that were foveal involving and 1 (7.1%) participant had foveal sparing GA. The mean GA area was 10.02 mm². The FAF patterns in the junction zone included banded (8 participants [57.1%]), diffuse (5 participants [35.7%]), and focal (1 participant [7.1%]). Two participants (11.8%) had CNV by FA, 1 participant could not be graded.

### Optical Coherence Tomography (OCT) Features

### Study Eye

Overall, OCT features were consistent with participants with GA. The mean OCT central subfield thickness was 182.41 μm across all participants. 4 participants had pigment epithelial detachments (PEDs), with mean thickness of 143.25 μm. No participants had intraretinal, subretinal fluid, or CNV by OCT. Mean drusen volume within a 5 mm circle was 0.05 mm³ across all participants.

### Fellow Eye

The mean OCT central subfield thickness was 211.96 μm across all participants. Eight participants had PED with mean thickness of 150.63 um. One (1) participant had subretinal fluid and 2 participants had intraretinal fluid. Twelve (70.6%) participants did not have CNV on optical coherence tomography angiography (OCTA). Four (4) participants had CNV Type 1 on OCTA and 1 participant had CNV which could not be graded. Mean drusen volume by OCT in the central 5 mm circle was 0.11 mm³ across all participants.

### Prior and Concomitant Therapy

The most common prior therapeutic categories by WHO ATC classification included vitamins (94.1%), diuretics (58.8%), lipid modifying agents (58.8%), and antithrombotic agents (52.9%). The most common prior ocular therapy was Ophthalmologicals (29.4%) with the PT macrogol 400; propylene glycol (17.6%).

The most common concomitant therapeutic categories by WHO ATC classification included therapies for the Cardiovascular system (58.8%) (such as diuretics, lipid modifying agents and renin-angiotensin system anti-hypertensives), Systemic anti-infectives (52.9% such as systemic antibacterials), and Ophthalmologicals (52.9% with the most common PT of Artificial Tears [umbrella Term] [23.5%]).

### Medical History

All participants had a diagnosis of AMD with GA in the study eye as per the inclusion criteria. The most common non-ocular medical histories were hypertension (76.5%) and arthritis (58.8%). The most common ocular procedure performed in both the study eyes (82.4%) and fellow eyes (82.4%) of participants was cataract extraction with posterior chamber intraocular lens implantation.

### Study Intervention

The study intervention(s) administered to participants is outlined in Table 6. The study intervention was administered to the eye which met the I/E criteria and had the worse visual acuity. Three participants received Dose 1 (3.56×10¹⁰ DRP), 3 participants received Dose 2 (1.07×10¹¹ DRP), and 11 participants received Dose 3 (3.56×10¹¹ DRP).

TABLE 6
Study Intervention(s) Administered

| Arm Name | Cohort 1 | Cohort 2 | Cohort 3 | Cohort 4^{a} |
|---|---|---|---|---|
| Intervention Name | AAVCAGsCD59 | AAVCAGsCD59 | AAVCAGsCD59 | AAVCAGsCD59 |
| Type | Biologic | Biologic | Biologic | Biologic |
| Dose Formulation | Other | Other | Other | Other |
| Unit Dose Strength(s) | 3.56 × 10¹⁰ DRP | 1.071 × 10¹¹ DRP | 3.56 × 10¹¹ DRP | 1.071 × 10¹² DRP |
| Dosage Level(s) | 0.1 mL single | 0.1 mL single | 0.1 mL single | 0.1 mL single |
| | injection | injection | injection | injection |
| Route of | Intravitreal | Intravitreal | Intravitreal | Intravitreal |
| Administration | injection | injection | injection | injection |
| Use | Experimental | Experimental | Experimental | Experimental |
| Investigational | Yes | Yes | Yes | Yes |
| Medicinal Product | | | | |
| (IMP) | | | | |
| Non-Investigational | No | No | No | No |
| Medicinal Product | | | | |
| (NIMP) | | | | |
| Sourcing | Delivered to | Delivered to | Delivered to | Delivered to |
| | trial site from | trial site from | trial site from | trial site from |
| | the compounding | the compounding | the compounding | the compounding |
| | pharmacy | pharmacy | pharmacy | pharmacy |
| Packaging and | Study intervention | Study intervention | Study intervention | Study intervention |
| Labeling | will be provided | will be provided | will be provided | will be provided |
| | in a capped 1 mL | in a capped 1 mL | in a capped 1 mL | in a capped 1 mL |
| | slip tip | slip tip | slip tip | slip tip |
| | polypropylene | polypropylene | polypropylene | polypropylene |
| | syringe pre- | syringe pre- | syringe pre- | syringe pre- |
| | diluted in a 0.2 | diluted in a 0.2 | diluted in a 0.2 | diluted in a 0.2 |
| | mL volume. | mL volume. | mL volume. | mL volume. |
| | Not in child | Not in child | Not in child | Not in child |
| | resistant | resistant | resistant | resistant |
| | packaging | packaging | packaging | packaging |
| Delivery Instructions^{b} | Delivered to | Delivered to | Delivered to | Delivered to |
| | site on ice | site on ice | site on ice | site on ice |
| Food/Fasting | N/A | N/A | N/A | N/A |
| Requirement^{b} | | | | |
| Current/Former | N/A | N/A | N/A | N/A |
| Name(s) or Alias(es)^{a} | | | | |

^{a}Highest dose of AAVCAGsCD59 was not administered to any study participants as sponsor ended enrollment after Cohort 3 due to slow recruitment.
^{b}Labels contained information to meet the applicable regulatory requirements.

### Distribution of AAVCAGsCD59 Vector

Quantitative polymerase chain reaction (PCR) was performed to detect the presence of AAVCAGsCD59 RNA in the serum at baseline, Day 7, Week 4, Week 12, and Week 26. Of the 17 participants, 3 participants had quantifiable sCD59 in the serum at any timepoint, with 1 additional participant having detectable AAVCAGsCD59 RNA below the lower limit of quantification (LLOQ) at a single timepoint. None of the participants had detectable sCD59 in the serum at baseline. Two (2) participants had quantifiable values of sCD59 at Day 7, which went below the LLOQ by Week 4. One participant had quantifiable levels of sCD59 at Week 4, which was undetectable by Week 12. All participants were in the high dose cohort.

### AAV2 Serum Neutralizing Antibody (NAb) Titers

AAV2 neutralizing antibody titers were detected in all participants at baseline and were highly variable (range 1:5.10 to 1:50819.74). Seven (7) participants had baseline titers less than 1:100, 5 participants had titers between 1:100 and 1:10000, and 5 participants had titers greater than 1:10,000. Nine (9) out of 17 participants had a 4-fold increase in baseline with titers at any time point (termed “treatment-boosted NAbs”). One participant had one timepoint over 4-fold from baseline, but subsequent tiers were below 4-fold of baseline (termed “transient positive”). There was no apparent relationship between incidence of intraocular inflammation and either high baseline AAV2 neutralizing titers or change from baseline AAV2 neutralizing titers after study intervention.

### Anti-sCD59 Serum Antibody Titers

No participants had serum anti-sCD59 antibodies either at baseline or any other measurement during the study.

### Study Assessments/Measurements

Safety assessments included the following:
Adverse Events (AEs) and Serious Adverse Events (SAEs);
Physical examinations and vital signs;
Clinical laboratory measures including hematology, liver function tests, renal function tests, blood chemistry, urinalysis, and pregnancy test;
Prior and concomitant medications;
Distance visual acuity testing: Best Corrected Visual Acuity (BCVA) using the Early Treatment Diabetic Retinopathy Study (ETDRS) visual acuity chart;
Intraocular Pressure (IOP);
Biomicroscopy;
Dilated examination of lens, retina & fovea;
Spectral domain optical coherence tomography (SD-OCT);
Spectral domain optical coherence tomography angiography (SD-OCTA);
Fundus autofluorescence (FAF) imaging;
Color Fundus Photograph (CFP);
Fluorescein Angiography (FA);
Serum anti-AAV2 antibody titer;
Serum AAVCAGsCD59 vector distribution;
Serum anti-sCD59 antibody; and
Aqueous sCD59 levels.

### Evaluation of Response to Study Intervention

### Adverse Events

Table 7 summarizes the overall systemic treatment emergent adverse events (TEAEs) by dose level. Overall, 16 (94.1%) participants experienced 1 or more TEAEs, 1 participant (5.9%) died during the study, and 9 (52.9%) participants experienced 1 or more SAEs. None of the systemic TEAEs, systemic SAEs, or the death were considered related to treatment intervention.

TABLE 7
Overall Summary of Systemic TEAEs

| | Cohort 1 | Cohort 2 | Cohort 3 | | Total | |
|---|---|---|---|---|---|---|
| Analysis set: | 3 | 3 | 11 | | 17 | |
| Safety | | | | | | |
| Analysis set | | | | | | |
| Subjects with 1 | | | | | | |
| or more: | | | | | | |
| AEs | 3 (100.0%) | 3 (100.0%) | 10 | (90.9%) | 16 | (94.1%) |
| Related AEs ^{a} | 0 | 0 | 0 | | 0 | |
| AEs leading to | 0 | 0 | 1 | (9.1%) | 1 | (5.9%) |
| death^{b} | | | | | | |
| Serious AEs | 1 (33.3%) | 2 (66.7%) | 6 | (54.5%) | 9 | (52.9%) |

| | | | | |
|---|---|---|---|---|
| Related serious | 0 | 0 | 0 | 0 |
| AEs | | | | |
| AEs leading to | 0 | 0 | 0 | 0 |
| discontinuation | | | | |
| from the study | | | | |

AE = adverse event,
DRP = DNase-Resistant Particles
^{a} An AE is assessed by the investigator as possibly or definitely related to study agent
^{b}AEs leading to death are based on AE outcome of Fatal
Cohort 1 = 3.56 × 10¹⁰ DRP;
Cohort 2 = 1.071 × 10¹¹ DRP;
Cohort 3 = 3.56 × 10¹¹ DRP

### Incidence of Systemic Adverse Events by System Organ Class

The most frequently reported preferred term (PT) for systemic (non-ocular) TEAEs were urinary tract infection (5 participants [29.4%]), fall (5 participants [29.4%]), and bradycardia (3 participants [17.6%]). The remaining PTs were reported by 1 or 2 participants. Table 8 summarizes the systemic TEAEs reported in at least 2 participants. There was no clustering of AEs within 1 or more system organ class (SOC). None of these TEAEs were related to the study intervention.

TABLE 8
Summary of Systemic TEAEs Reported in at Least 2 Participants
by System Organ Class and Preferred Term

| | Cohort 1 | Cohort 2 | Cohort 3 | | Total |
|---|---|---|---|---|---|
| Analysis set: | 3 | 3 | 11 | | 17 |
| Safety | | | | | |
| Analysis set | | | | | |
| Subjects with one | 2 (66.7%) | 3 (100.0%) | 8 | (72.7%) | 13 (76.5%) |
| or more AEs | | | | | |
| System organ class | | | | | |
| Preferred term | | | | | |
| Infections and | 1 (33.3%) | 1 (33.3%) | 3 | (27.3%) | 5 (29.4%) |
| infestations | | | | | |
| Urinary tract | 1 (33.3%) | 1 (33.3%) | 3 | (27.3%) | 5 (29.4%) |
| infection | | | | | |
| Cardiac disorders | 1 (33.3%) | 2 (66.7%) | 2 | (18.2%) | 5 (29.4%) |
| Atrial fibrillation | 0 | 1 (33.3%) | 1 | (9.1%) | 2 (11.8%) |
| Bradycardia | 1 (33.3%) | 1 (33.3%) | 1 | (9.1%) | 3 (17.6%) |
| Gastrointestinal | 0 | 0 | 2 | (18.2%) | 2 (11.8%) |
| disorders | | | | | |
| Large intestine | 0 | 0 | 2 | (18.2%) | 2 (11.8%) |
| polyp | | | | | |
| Respiratory, | 0 | 0 | 2 | (18.2%) | 2 (11.8%) |
| thoracic and | | | | | |
| mediastinal | | | | | |
| disorders | | | | | |
| Chronic obstructive | 0 | 0 | 2 | (18.2%) | 2 (11.8%) |
| pulmonary disease | | | | | |
| Vascular disorders | 0 | 0 | 2 | (18.2%) | 2 (11.8%) |
| Hypertension | 0 | 0 | 2 | (18.2%) | 2 (11.8%) |
| Injury, poisoning | 2 (66.7%) | 2 (66.7%) | 1 | (9.1%) | 5 (29.4%) |
| and procedural | | | | | |
| complications | | | | | |
| Fall | 2 (66.7%) | 2 (66.7%) | 1 | (9.1%) | 5 (29.4%) |

| | | | | |
|---|---|---|---|---|
| Nervous system | 2 (66.7%) | 0 | 0 | 2 (11.8%) |
| disorders | | | | |
| Headache | 2 (66.7%) | 0 | 0 | 2 (11.8%) |

Key:
AE = Adverse event,
DRP = DNase-Resistant Particles
AEs are coded using MedDRA Version 23.0.
Cohort 1 = 3.56 × 10¹⁰ DRP;
Cohort 2 = 1.071 × 10¹¹ DRP;
Cohort 3 = 3.56 × 10¹¹ DRP

The majority of TEAEs were mild in severity. Nine (9) TEAEs were severe and those arose in the following SOCs: cardiac disorders: 2 events; gastrointestinal disorders: 1 event; injury, poisoning and procedural complications: 1 event; neoplasms benign, malignant, and unspecified: 3 events; respiratory, thoracic and mediastinal disorders: 1 event; vascular disorders: 1 event. One (1) participant died during the study on Day 244. This participant had an ongoing medical history of white blood cell disorder since 2016. The cause of death was reported as leukemia and was determined by the investigator to not be related to the study intervention.

### Serious Adverse Events

Nine (9) participants (52.9%) experienced 1 or more serious adverse events (SAEs). None of the SAEs were considered related to the study intervention. The SAEs were not clustered in a particular SOC. The SOCs with the most frequently reported SAEs included neoplasms benign, malignant and unspecified; injury, poisoning and procedural complications; and cardiac disorders with each SOC having 3 participants (17.6%). No TEAEs led to discontinuation of the study.

### Ocular Adverse Events

### Study Eye

Thirteen (13) participants (76.5%) experienced ocular TEAEs in the study eye (Table 9). None of these TEAEs were considered SAEs. Nine (9) of the AEs had not resolved by the end of the study. All of the ocular TEAEs in the study eye were mild in severity, except for 1 moderate AE (basal cell carcinoma [reported term: basal cell on lower right eyelid]). The number of TEAEs in the study eye were balanced across the treatment groups. Twelve (12) participants (70.6%) reported study eye TEAEs in the eye disorders SOC. Vitritis was reported in 4 participants (23.5%; all in Cohort 3) and anterior chamber inflammation was reported in 1 participant (5.9%). Intraocular pressure increase was reported in 2 participants (11.8%). Retinal hemorrhage was reported in 2 participants (11.8%). The remainder of PTs occurred in only 1 participant. Reduced Visual acuity was reported in 3 participants (17.6%; all in Cohort 3) (Table 10). None of the participants that reported the reduced visual acuity AE had a clinically-significant ≥15 letter loss in the study eye at 2 consecutive visits at the time of the AE.

Six (6) participants (35.3%) reported an ocular adverse event in the study eye that was considered by the investigator to be related to study intervention. Five (5) of the participants were in Cohort 3 and 1 participant was in Cohort 2. The PTs for the related events were anterior chamber inflammation (1 participant), optic nerve disorder (1 participant) and vitritis (4 participants) (Table 11). The anterior chamber inflammation (reported term: mild post-injection anterior inflammation) and optic nerve disorder (reported term: worsening of cup to disc ratio oculus uterque [OU]) were in the same participant. All TEAEs in the study eye were mild, except 1 moderate TEAE on the study eyelid (basal cell carcinoma).

### Bilateral Eyes

There were 5 TEAEs that were present in both eyes. Two (2) participants had bilateral ocular adverse events of optic nerve disorder (reported term: worsening of cup to disc ratio). One (1) participant had allergic conjunctivitis, 1 participant had seasonal allergy, and 1 participant had an adverse event of cataract (reported term: worsening of cataract).

TABLE 9
Overall Summary of Study Eye TEAEs

| | Cohort 1 | Cohort 2 | Cohort 3 | Total |
|---|---|---|---|---|
| Analysis set: | 3 | 3 | 11 | 17 |
| Safety Analysis set | | | | |
| Subjects with 1 | | | | |
| or more: | | | | |
| AEs | 2 (66.7%) | 3 (100.0%) | 8 (72.7%) | 13 (76.5%) |
| Related AEs ^{a} | 0 | 1 (33.3%) | 5 (45.5%) | 6 (35.3%) |
| AEs leading | 0 | 0 | 0 | 0 |
| to death ^{b} | | | | |
| Serious AEs | 0 | 0 | 0 | 0 |
| Related serious | 0 | 0 | 0 | 0 |
| AEs | | | | |
| AEs leading to | 0 | 0 | 0 | 0 |
| discontinuation | | | | |
| from the study | | | | |

AE = adverse event,
DRP = DNase-Resistant Particles
^{a a} An AE is assessed by the investigator as possibly and definitely related to study agent
^{b b} AEs leading to death are based on AE outcome of Fatal
Cohort 1 = 3.56 × 10¹⁰ DRP;
Cohort 2 = 1.071 × 10¹¹ DRP;
Cohort 3 = 3.56 × 10¹¹ DRP

TABLE 10
Summary of Study Eye TEAEs by System Organ Class and Preferred Term

| | Cohort 1 | Cohort 2 | Cohort 3 | | Total | |
|---|---|---|---|---|---|---|
| Analysis set: | 3 | 3 | 11 | | 17 | |
| Safety | | | | | | |
| Analysis set | | | | | | |
| Subjects with | 2 (66.7%) | 3 (100.0%) | 8 | (72.7%) | 13 | (76.5%) |
| one or more AEs | | | | | | |
| System organ class | | | | | | |
| Preferred term | | | | | | |
| Eye disorders | 1 (33.3%) | 3 (100.0%) | 8 | (72.7%) | 12 | (70.6%) |
| Vitritis | 0 | 0 | 4 | (36.4%) | 4 | (23.5%) |
| Visual acuity | 0 | 0 | 3 | (27.3%) | 3 | (17.6%) |
| reduced | | | | | | |
| Cataract | 0 | 0 | 1 | (9.1%) | 1 | (5.9%) |
| Conjunctival | 0 | 0 | 1 | (9.1%) | 1 | (5.9%) |
| hemorrhage | | | | | | |
| Ectropion | 0 | 0 | 1 | (9.1%) | 1 | (5.9%) |
| Optic nerve | 0 | 1 (33.3%) | 1 | (9.1%) | 2 | (11.8%) |
| disorder | | | | | | |
| Retinal hemorrhage | 0 | 0 | 1 | (9.1%) | 1 | (5.9%) |
| Vitreous floaters | 0 | 0 | 1 | (9.1%) | 1 | (5.9%) |
| Anterior chamber | 0 | 1 (33.3%) | 0 | | 1 | (5.9%) |
| inflammation | | | | | | |
| Conjunctivitis | 1 (33.3%) | 0 | 0 | | 1 | (5.9%) |
| allergic | | | | | | |
| Dry eye | 0 | 1 (33.3%) | 0 | | 1 | (5.9%) |
| Posterior capsule | 0 | 1 (33.3%) | 0 | | 1 | (5.9%) |
| opacification | | | | | | |
| Vitreous | 0 | 1 (33.3%) | 0 | | 1 | (5.9%) |
| hemorrhage | | | | | | |
| Injury, poisoning | 0 | 0 | 1 | (9.1%) | 1 | (5.9%) |
| and procedural | | | | | | |
| complications | | | | | | |
| Corneal abrasion | 0 | 0 | 1 | (9.1%) | 1 | (5.9%) |
| Investigations | 0 | 1 (33.3%) | 1 | (9.1%) | 2 | (11.8%) |
| Intraocular | 0 | 1 (33.3%) | 1 | (9.1%) | 2 | (11.8%) |
| pressure increased | | | | | | |

| | | | | | |
|---|---|---|---|---|---|
| Immune system | 1 (33.3%) | 0 | 0 | 1 | (5.9%) |
| disorders | | | | | |
| Seasonal allergy | 1 (33.3%) | 0 | 0 | 1 | (5.9%) |
| Neoplasms benign, | 1 (33.3%) | 0 | 0 | 1 | (5.9%) |
| malignant and | | | | | |
| unspecified (incl | | | | | |
| cysts and polyps) | | | | | |
| Basal cell | 1 (33.3%) | 0 | 0 | 1 | (5.9%) |
| carcinoma | | | | | |

Key:
AE = adverse event,
DRP = DNase-Resistant Particles
Note:
Participants were counted only once for any given event
AEs are coded using MedDRA Version 23.0.
Cohort 1 = 3.56 × 10¹⁰ DRP;
Cohort 2 = 1.071 × 10¹¹ DRP;
Cohort 3 = 3.56 × 10¹¹ DRP

TABLE 11
Summary of Participants with Related Study Eye TEAEs by Preferred Term

| | Cohort 1 | Cohort 2 | Cohort 3 | Total |
|---|---|---|---|---|
| Subjects treated | 3 | 3 | 11 | 17 |
| Subjects with one | 0 | 1 (100.0%) | 5 (100.0%) | 6 (100.0%) |
| or more TEAEs | | | | |
| Dictionary-Derived | | | | |
| Term | | | | |
| Anterior chamber | 0 | 1 (100.0%) | 0 | 1 (16.7%) |
| inflammation | | | | |
| Optic nerve disorder | 0 | 0 | 1 (20.0%) | 1 (16.7%) |
| Vitritis | 0 | 0 | 4 (80.0%) | 4 (66.7%) |

Key:
AE = adverse event,
DRP = DNase-Resistant Particles
Cohort 1 = 3.56 × 10¹⁰ DRP;
Cohort 2 = 1.071 × 10¹¹ DRP;
Cohort 3 = 3.56 × 10¹¹ DRP
Adverse events are coded using MedDRA Version 23.0.
Note:
Subjects are counted only once for any given event

### Fellow Eye

Nine (9) participants (52.9%) experienced ocular TEAEs in the fellow eye. None of the TEAEs were SAEs. One (1) participant (Cohort 3) reported an adverse event in the fellow eye of optic nerve disorder that was considered by the investigator to be possibly related to study treatment. Seven (7) participants (41.2%) reported fellow eye TEAEs in the eye disorders SOC. The most frequently reported PTs were dry eye (2 participants [11.8%]) and optic nerve disorder OU (2 participants [11.8%]). Both reported terms for optic nerve disorder was worsening of cup to disc ratio. Both of the adverse events of optic nerve disorder were bilateral events. All the remaining PTs were reported only once. There were no ocular inflammation AEs in the fellow eye.

There was 1 severe TEAE in the fellow eye (PT: skin disorder [reported term: superior scaly, erythematous lesion on upper lid OS]) and 1 moderate TEAE in the fellow eye (PT: retinal tear [reported term: horseshoe tear of retina]).

### Ocular Adverse Events of Interest

The following AEs were deemed to be of interest:
Endophthalmitis;
Intraocular inflammation;
Intraocular pressure increase;
Ocular hemorrhage;
Cataract; and
Retinal structural change, deposit and degeneration.

These categories consisted of specified PTs determined by the sponsor.

In addition to the above categories, ocular TEAE preferred terms were reviewed and the following adverse events of interest were included:
optic nerve disorder;
retinopathy;
neovascular AIMD
retinal artery embolism; and
vitreous floaters.

### Study Eye

Thirteen (13) AEs of interest in 9 participants were experienced in the study eye (Table 12). None of these events occurred in Cohort 1. Five (5) of these AEs were categorized as intraocular inflammation (29.4%) with PTs of anterior chamber inflammation (Cohort 2; 1 event; onset Day 27; duration of 24 days) and vitritis (4 events; all in Cohort 3). The 4 events of vitritis had onset days of Day 45 (duration 47 days), Day 20 (duration 346 days), Day 34 (duration 170 days), and Day 29 (unresolved since participant died during the trial). All the intraocular inflammation AEs were considered by the investigator to be possibly related to study treatment and were mild in severity. The event of anterior chamber inflammation was treated with difluprednate from Days 28 to 70 and 1 event of vitritis was treated with prednisolone opthalmological from Days 57 to 85. The other 3 events of vitritis did not result in the participant being treated.

Two (2) AEs in 2 participants of transient intraocular pressure were reported, both of which occurred either the day of or the day after the onset of an intraocular inflammation adverse event. Both were mild in severity and considered by the investigator to be unrelated to study intervention. Onset of these events occurred on Day 28 and Day 34. One (1) participant experienced an increase in IOP from 12 mmHg on Day 15 to 21 mmHg on Day 28. The pressure remained the same on Day 21 but decreased to 14 mmHg by Day 34. The participant did not receive any treatment for the increase in ocular pressure. Another participant experienced an increase in IOP from 17 mmHg on Day 9 to 28 mmHg on Day 34. The pressure decreased to 10 mmHg on Day 43. The participant received brimonidine tartate/timolol ophthalmic drops from Day 34 to Day 42. Both participants recovered within 10 days from the transient intraocular pressure increase and both participants eventually recovered from the intraocular inflammation AEs.

Three (3) AEs in 2 participants were categorized as ocular hemorrhage. All occurred in the study eye, were mild in severity, and considered by the investigator to be unrelated to study intervention. One (1) of the ocular hemorrhages was a retinal hemorrhage with onset at Day 727 and as of the end of the study, the participant had not yet recovered. The other 2 ocular hemorrhages were in the same participant and were vitreous hemorrhages with onset at Day 2. The reported terms for these 2 events were: small vitreous hemorrhage right eye (resolved on Day 14) and 16 VA letter loss secondary to small vitreous hemorrhage (resolved on Day 28) and both coded to a PT of vitreous hemorrhage. While vitreous hemorrhage was considered unrelated to study intervention, relationship to study procedure of AEs was not captured in this study. However, vitreous hemorrhage is an AE of intravitreal injections as observed in clinical practice and clinical trials.

One (1) AE was categorized as cataract. This AE occurred on Day 120, was mild in severity and considered by the investigator to be unrelated to study intervention. The participant had an ongoing ocular medical history of cataract since 2009. The reported term for the event was worsening of cataract and remained unresolved at the end of the study.

There were 2 AEs of worsening of cup to disc ratio (PT: optic nerve disorder) in 2 participants. Both participants experienced this adverse event in both study eyes and fellow eyes. One (1) participant experienced the adverse event with an onset at Day 281, was considered by the investigator to be not related to study intervention and was unresolved at the end of the study. The other participant experienced the adverse event with an onset at Day 244, was considered by the investigator to be possibly related to study intervention and was unresolved at the end of the study. This participant did not experience any other concurrent ocular adverse event of interest. There were no cases of endophthalmitis in the study eye.

TABLE 12
Summary of Study Eye TEAEs of Interest by
AE of Interest Category and Preferred Term

| | Cohort 1 | Cohort 2 | Cohort 3 | | Total | |
|---|---|---|---|---|---|---|
| Analysis set: | 3 | 3 | 11 | | 17 | |
| Safety Analysis set | | | | | | |
| Subjects with one | 0 | 2 (66.7%) | 7 | (63.6%) | 9 | (52.9%) |
| or more AEoIs | | | | | | |
| AE of Interest | | | | | | |
| Category | | | | | | |
| Preferred term | | | | | | |
| Intraocular | 0 | 1 (33.3%) | 4 | (36.4%) | 5 | (29.4%) |
| Inflammation | | | | | | |
| Vitritis | 0 | 0 | 4 | (36.4%) | 4 | (23.5%) |
| Anterior chamber | 0 | 1 (33.3%) | 0 | | 1 | (5.9%) |
| inflammation | | | | | | |
| Other | 0 | 1 (33.3%) | 2 | (18.2%) | 3 | (17.6%) |
| uncategorized AEoI | | | | | | |
| Optic nerve | 0 | 1 (33.3%) | 1 | (9.1%) | 2 | (11.8%) |
| disorder | | | | | | |
| Vitreous floaters | 0 | 0 | 1 | (9.1%) | 1 | (5.9%) |
| Cataract | 0 | 0 | 1 | (9.1%) | 1 | (5.9%) |
| Cataract | 0 | 0 | 1 | (9.1%) | 1 | (5.9%) |
| Ocular | 0 | 1 (33.3%) | 1 | (9.1%) | 2 | (11.8%) |
| hemorrhage | | | | | | |
| Retinal | 0 | 0 | 1 | (9.1%) | 1 | (5.9%) |
| hemorrhage | | | | | | |
| Vitreous | 0 | 1 (33.3%) | 0 | | 1 | (5.9%) |
| hemorrhage | | | | | | |
| Transient | 0 | 1 (33.3%) | 1 | (9.1%) | 2 | (11.8%) |
| Intraocular | | | | | | |
| pressure increase | | | | | | |
| Intraocular | 0 | 1 (33.3%) | 1 | (9.1%) | 2 | (11.8%) |
| pressure increased | | | | | | |

Key:
AEoIs = Adverse events of Interest,
DRP = DNase-Resistant Particles
Note:
Subjects are counted only once for any given event.
Adverse events are coded using MedDRA Version 23.0.
Cohort 1 = 3.56 × 10¹⁰ DRP;
Cohort 2 = 1.071 × 10¹¹ DRP;
Cohort 3 = 3.56 × 10¹¹ DRP

### Fellow Eye

Seven (7) AEs of interest in 4 participants were reported in the fellow eye. Six (6) of the 7 were mild in severity and 1 was moderate in severity (PT retinal tear). All of the AEs, except for 1, were deemed by the investigator to not be related to study intervention. An AE of worsening of cup to disc ratio (PT: optic nerve disorder) with onset at Day 244 was considered by the investigator to possibly be related to study intervention. This AE was still ongoing at end of study. This participant also had the same AE in the study eye. Another AE of worsening of cup to disc ratio in both eyes was experienced by another participant. The PTs of the remaining AEs occurring in one participant each were: retinal artery embolism, neovascular age-related macular degeneration, retinopathy and cataract. One (1) participant had an AE of retinal artery embolism (reported term: Hollenhorst plaque) with an onset at Day 457. The event was mild in severity, not considered related by the investigator and participant had not recovered by the end of the study. No cases of endophthalmitis in the fellow eye were reported.

### Clinical Laboratory Evaluation

There were no clinically significant changes in laboratory data. The majority of the laboratory values were Grade 0 or Grade 1 (per NCI-CTCAE grading). One (1) participant (Cohort 3) had a toxicity Grade 4 neutropenia at Week 4. This participant had toxicity Grade 3 neutropenia at baseline, Day 7, and Week 12. Neutrophil levels returned to within normal limits at end of study (Day 190). In addition, this participant had a toxicity Grade 2 or 3 lymphocytopenia from baseline through end of study (Day 190). This participant had a fatal AE of leukemia on Day 244 (Section 5.1.2.2).

### Other Safety Evaluations

### Vital Signs/Physical Evaluation

There were no clinically meaningful findings in the vital sign measurements from baseline over time in this study. There were no clinically significant changes in the physical examinations related to study intervention in this study.

### Intraocular Pressure (IOP)

Baseline IOP ranged from 8 to 21.50 mmHg for the study eye, and 9.5 to 21.50 mm Hg for the fellow eye. Mean change in IOP from baseline was less than 2 mm Hg for all cohorts in the study eye at Week 26 and Week 104. Mean change in IOP from baseline was less than 2.5 mm Hg for all cohorts in the fellow eye at Week 26 and Week 104. The highest mean change in IOP from baseline was 3 mm Hg from baseline for the study eye, and 2.5 mm Hg from baseline for the fellow eye.

Two (2) participants experienced a transient increase in IOP AEs of interest. Both participants experienced an intraocular inflammation AE of interest either the same day or the day prior to the increase in IOP adverse event. One of the participants received no treatment while the other participant received intraocular brimonidine tartate/timolol.

### Slit Lamp Biomicroscopy

In general, slit lamp biomicroscopy examination was consistent with the demographics of the participant population. There were no clinically-significant changes in slit lamp biomicroscopy related to study intervention except those noted in the above AEs of interest in either the study eye or fellow eye.

### Indirect/Dilated Ophthalmoscopy

There were no clinically significant changes in indirect/dilated ophthalmoscopy related to study intervention outside of the above-mentioned AEs of interest in either the study eye or fellow eye.

### Secondary Evaluations

### Rate of Growth of GA Lesions

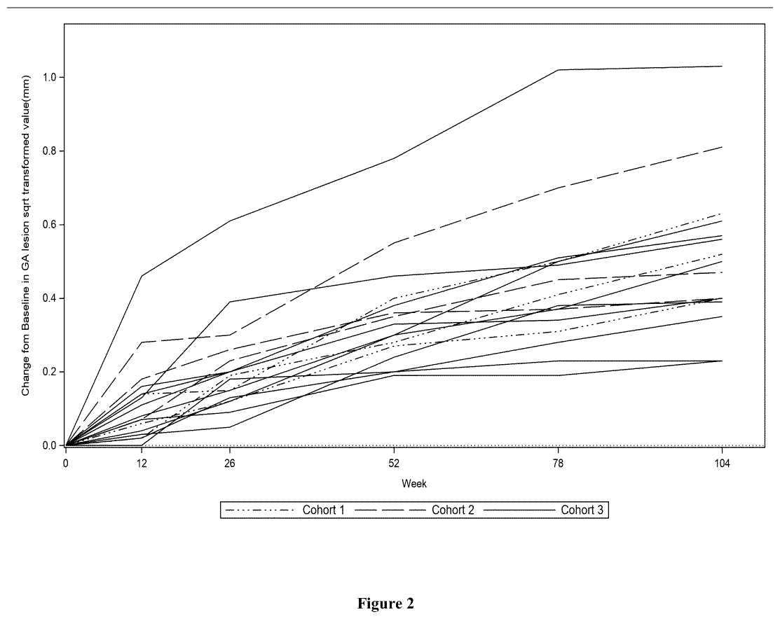

Individual participant data of change in GA growth by baseline in square root transformation of the study eye is presented in FIG. 2. Baseline GA lesion area in square root transformed value (mm) was 3.4 mm (range 2.85; 3.79) in Cohort 1, 3.373 mm (range 3.01; 3.58) in Cohort 2, and 3.222 mm (range 2.34; 4.42) in Cohort 3. Mean change in baseline at Week 26 was 0.153 mm in Cohort 1, 0.263 mm in Cohort 2, and 0.338 mm in Cohort 3. Mean change in baseline at Week 104 was 0.517 mm in Cohort 1, 0.56 mm in Cohort 2, and 0.487 mm in Cohort 3.

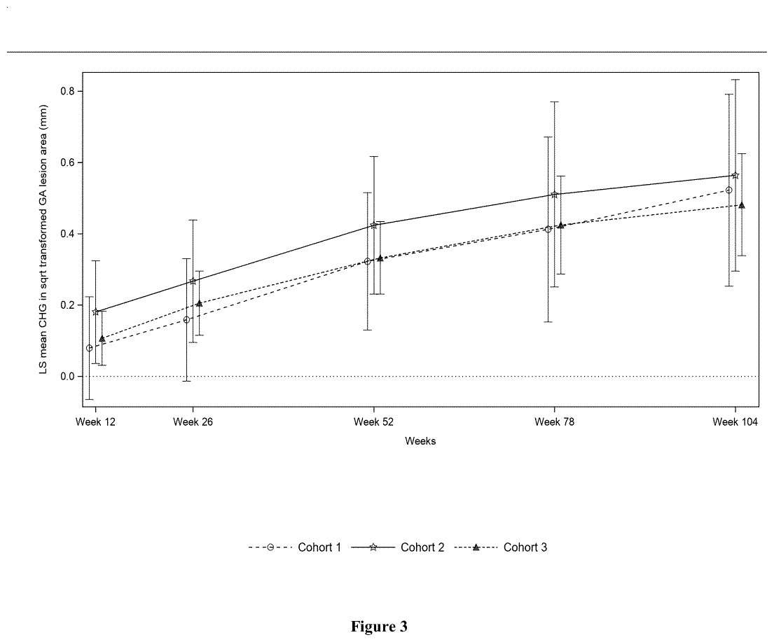

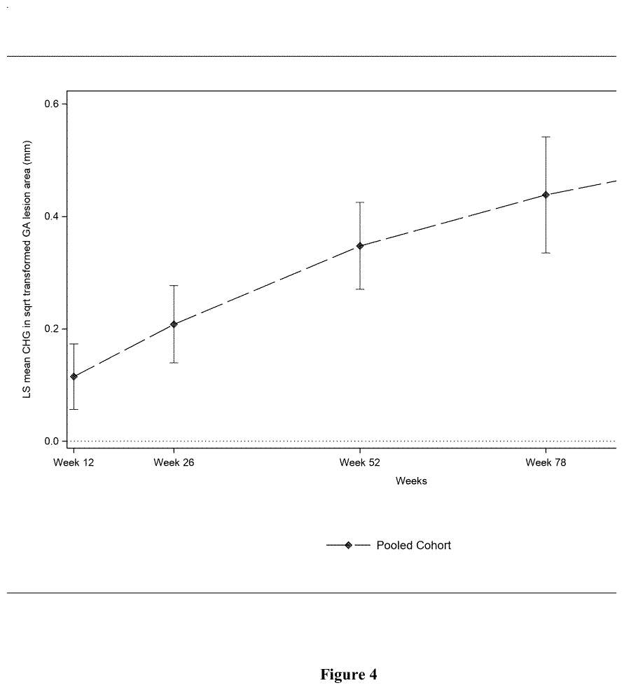

A Mixed model repeated measures (MMRM) was used to analyze GA growth over time for each of the dosing arms as well as pooled analysis of all cohorts (FIG. 3 and FIG. 4). This model incorporated baseline lesion size, dose level, selected visits, and dose level (cohort) by visit interaction as covariates. Given the limited sample size, no formal analysis was performed looking at differences in GA growth by dosing arms. There were no apparent differences in GA growth by dose.

Baseline GA lesion area in square root transformed value of fellow eyes were 2.473 mm (range 0.85; 3.54) in Cohort 1, 3.52 mm (range 3.41; 3.63) in Cohort 2, and 3.041 mm (range 1.25; 4.76) for Cohort 3. Mean change in baseline at Week 104 ranged from 0.527 to 1.124 mm across the 3 cohorts.

### Change in Area of GA Lesion Over Time

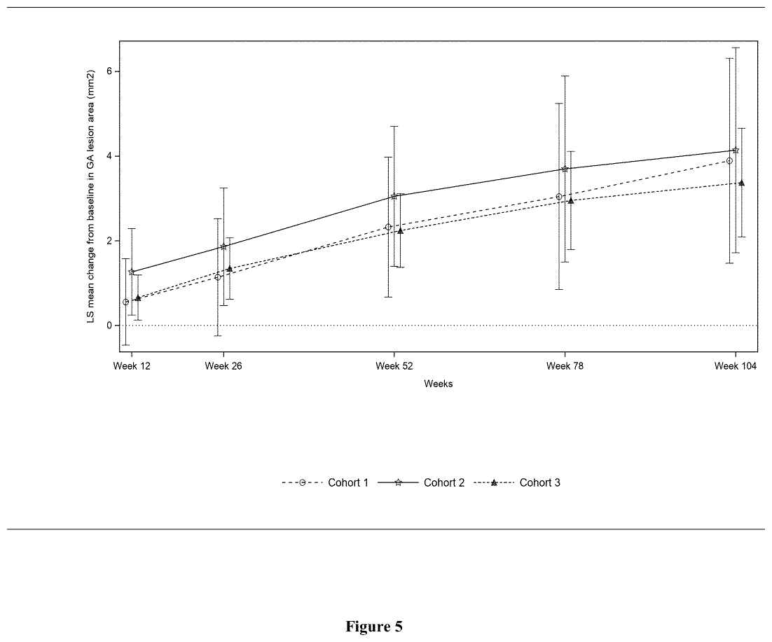

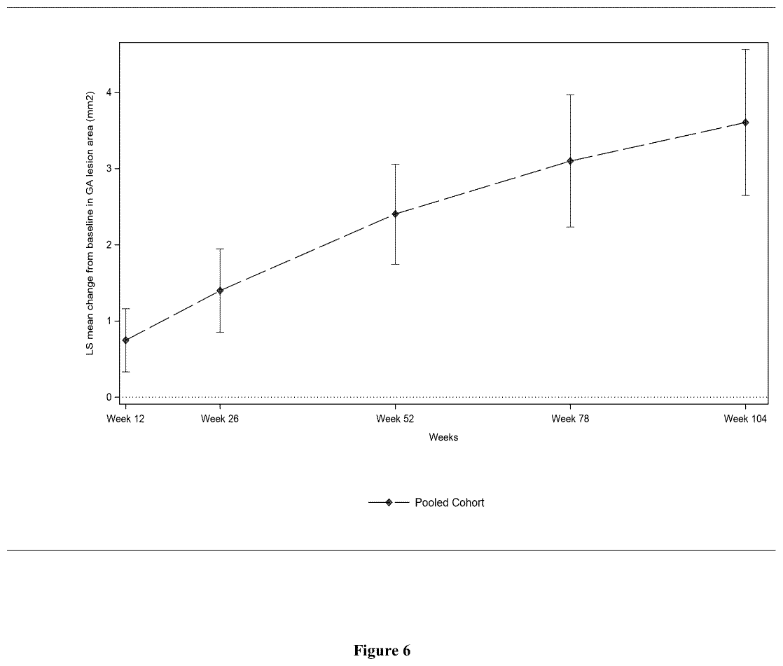

Baseline GA lesion area was similar across dosing arms. Mean baseline GA lesions area was 11.72 mm² (range 8.15;14.39) for Cohort 1, 11.467 mm² (9.08; 12.84) for Cohort 2, and 10.865 mm² (5.47; 19.50) for Cohort 3. Percent change from baseline at Week 26 was 9.235 for Cohort 1, 16.037 for Cohort 2, and 14.888 for Cohort 3. Percent change from baseline at Week 104 was 32.572 for Cohort 1, 35.830 for Cohort 2, and 34.406 in Cohort 3. Due to small numbers, there was no formal analysis of differences in dosing arms with rate of growth of GA. The MMRM estimating change in GA lesion size for each dosing cohort as well as for the pooled cohorts is shown in FIG. 5 and FIG. 6.

### Conversion of Dry AMD to Wet AMD

There were no participants in any of the cohorts who converted from dry AMD to wet AMD (defined as new presence of CNV) in the study eye, as assessed by FA, OCT, and OCTA. One (1) participant in Cohort 2 converted from dry AMD to wet AMD in the fellow eye on Day 83. This was believed to be due to the natural history of the disease and was not related to the study intervention.

### Drusen Volume

There were no clinically significant changes in drusen volume in the study eye or fellow eye across all participants.

### Visual Acuity

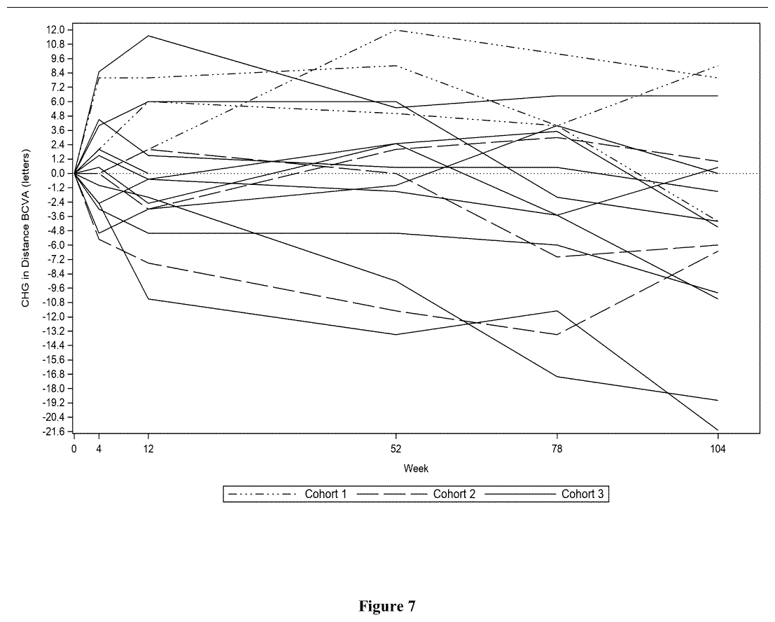

Individual participant data for visual acuity scores over time for the study eye is shown in FIG. 7. Of note, the mean baseline BCVA in ETDRS letters for Cohort 1 was lower than those for Cohort 2 and 3. Mean BCVA for Cohort 1 was 18.667 letters (range 13 to 26), 50.5 letters (48 to 53) for Cohort 2, and 38.727 letters (22.5 to 56.50) for Cohort 3. The mean change from baseline at Week 26 was +3.667 letters, −0.500 letters, and −1.636 letters in Cohorts 1, 2, 3, respectively. The change from baseline at Week 104 was +4.33 letters, −3.833 letters, and −6.40 letters in Cohorts 1, 2, and 3, respectively. No significant trends in differences in visual acuity between the 3 dose cohorts was observed.

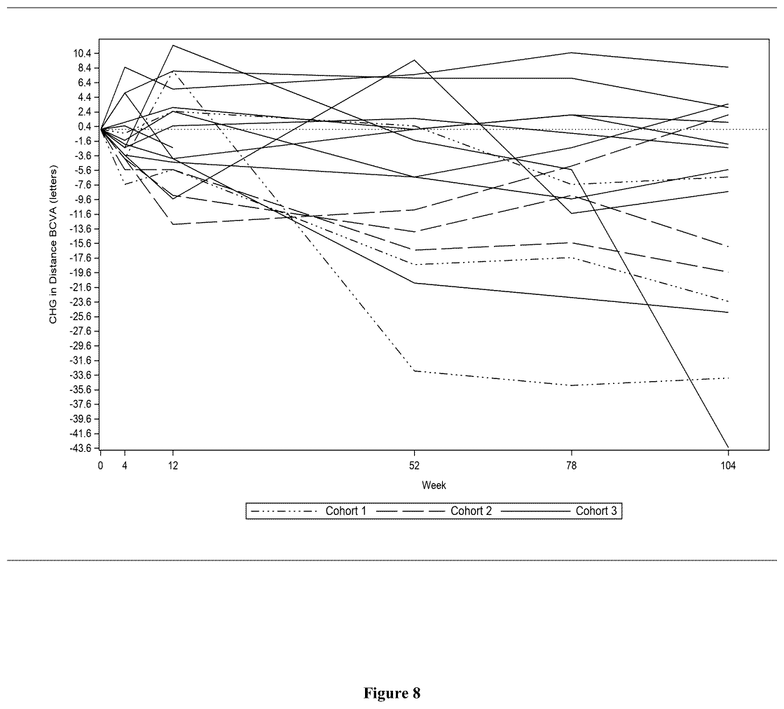

The mean baseline BCVA in the fellow eye was lower for Cohort 1 compared with Cohort 2 and 3. The mean baseline BCVA for the fellow eye for Cohort 1 was 44.000 letters (28.50 to 71.00), 59.167 letters (50.50 to 70.00) for Cohort 2 and 59.591 letters (35.50 to 78.00) for Cohort 3. The mean baseline BCVA change from baseline at Week 26 was −5.333 letters, −7.056 letters and −0.205 letters in Cohorts 1, 2, and 3, respectively. The mean BCVA change from baseline at Week 104 was −21.333 letters, −11.167 letters and −7.100 letters in Cohorts 1, 2 and 3, respectively. Individual participant data for visual acuity scores over time for the fellow eye is shown in (FIG. 8).

Number and percentage of participants with loss of ≥10, ≥15, ≥20, and ≥30 letters at least once in distance BCVA from baseline over time is listed in Table 13. In terms of clinically significant change in vision, there were 2 participants in the study eye that lost ≥15 letters at least once. No participants lost ≥30 letters in the study eye. In the fellow eye, 6 participants (2 in each cohort) lost ≥15 letters and 2 s lost ≥30 letters at least once.

There were no participants who gained ≥15 letters of vision in the study eye or the fellow eye, consistent with the natural history of the disease.

TABLE 13
Number And Percentage Of Patients Losing ≥10, ≥15,
≥20, And ≥30 Letters at One or More Visits In
Distance BCVA From Baseline Over Time; Full Analysis Set

| | Cohort 1 | Cohort 2 | Cohort 3 |
|---|---|---|---|
| Analysis set: Full Analysis | 3 | 3 | 11 |
| Study Eye | | | |
| Subjects losing >=10 letters | 0 | 1 (33.3%) | 4 (36.4%) |
| Subjects losing >=15 letters | 0 | 0 | 2 (18.2%) |
| Subjects losing >=20 letters | 0 | 0 | 1 (9.1%) |
| Subjects losing >=30 letters | 0 | 0 | 0 |
| Fellow Eye | | | |
| Subjects losing >=10 letters | 2 (66.7%) | 3 (100.0%) | 3 (27.3%) |
| Subjects losing >=15 letters | 2 (66.7%) | 2 (66.7%) | 2 (18.2%) |
| Subjects losing >=20 letters | 2 (66.7%) | 0 | 2 (18.2%) |
| Subjects losing >=30 letters | 1 (33.3%) | 0 | 1 (9.1%) |

Key:
BCVA = best corrected visual acuity,
DRP = DNase-Resistant Particles
Cohort 1 = 3.56 × 10¹⁰ DRP
Cohort 2 = 1.071 × 10¹¹ DRP
Cohort 3 = 3.56 × 10¹¹ DRP

### Exploratory Evaluations

Levels of Intraocular (Aqueous) sCD59 Protein

Aqueous levels of sCD59 were determined using a custom Western Blot assay using a commercially available anti-CD59 rabbit polyclonal antibody (Abcam, Cambridge UK, cat. no. ab124396). Aqueous was collected at baseline prior to injection as well as Week 8. No participants had detectable levels of aqueous sCD59 protein prior to study intervention administration. Aqueous sCD59 protein was detected in 5/17 participants at Week 8. Levels of protein were highly variable with a range of 250 ng/ml to 5719.4 ng/ml. All participants with detectable sCD59 protein were in the high dose cohort. Western Blot analysis was not available for 1 participant (unable to electrophorese for unknown reasons).

In sum, the 3 doses of AAVCAGsCD59 tested in this study were safe and well tolerated with no dose limiting toxicity. The most clinically significant AE related to study intervention was intraocular inflammation which occurred in 29.4% of participants. However, intraocular inflammation in all participants were mild and were either self-limited or resolved with topical steroids. There were no systemic TEAEs related to the study intervention, which is consistent with the low systemic exposure of AAV2CAGsCD59 observed in serum. Production of the gene therapy product, sCD59, could be detected in the aqueous of a subset of participants.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for treating age-related macular degeneration (AMD) in a subject, the method comprising administering a pharmaceutical composition into an AMD-affected eye of a subject by ocular injection, wherein the composition comprises a nucleic acid encoding a soluble CD59 (sCD59) protein operably linked to a promoter, wherein the nucleic acid encoding sCD59 is packaged into a delivery vector and wherein the administering results in expression and secretion of the sCD59 protein by cells of the AMD-affected eye and the expression results in treatment of AMD-affected cells in the AMD-affected eye.

2. The method according to claim 1, wherein the AMD is geographic atrophy (GA).

3. The method according to claim 1, wherein the ocular injection is an intravitreal injection.

4. The method according to claim 2, wherein the intravitreal injection is a single injection.

5. The method according to claim 1, wherein the delivery vector is an adeno-associated virus (AAV) vector.

6. The method according to claim 4, wherein the AAV vector is AAV2.

7. The method according to claim 1, wherein the promoter is a CAG promoter.

8. The method according to claim 1, wherein the pharmaceutical composition comprises a dose of viral particles selected from the group consisting of about 3.56×10¹⁰ DNAse-resistant particles (DRP), about 1.071×10¹¹ DRP, about 3.56×10¹¹ DRP and about 1.07×10¹² DRP.

9. A method of regulating a complement activity disorder in a subject, the method comprising contacting an affected cell of the subject with a pharmaceutical composition comprising a vector carrying a nucleotide sequence encoding a recombinantly engineered human soluble CD59 (sCD59) protein operably linked to a promoter sequence causing expression of the protein in the affected cell, wherein the sCD59 protein comprises at least one mutation resulting in loss of function of glycosylphosphatidylinositol (GPI) anchoring domain resulting in loss of membrane targeting and observing a physiological indicium of the complement activity disorder after the contacting, in comparison to an abnormal amount of the physiological indicium observed prior to the contacting, wherein a decrease after the contacting compared prior to the contacting is a positive indication that the affected cell is treated.

10. The method according to claim 9, wherein the complement activity disorder is GA.

11. The method according to claim 9, wherein the contacting is by intravitreal injection.

12. The method according to claim 11, wherein the intravitreal injection is a single injection.

13. The method according to claim 9, wherein the affected cell is a retinal cell.

14. The method according to claim 9, wherein the vector is AAV2.

15. The method according to claim 9, wherein the physiological indicium is best corrected visual acuity (BCVA).

16. The method according to claim 15, wherein BCVA is measured as mean change from baseline

17. The method according to claim 16, wherein mean change from baseline is −7.100 letters.

18. The method according to claim 9, wherein the pharmaceutical composition comprises a dose of viral particles selected from the group consisting of about 3.56×10¹⁰ DNAse-resistant particles (DRP), about 1.071×10¹¹ DRP, about 3.56×10¹¹ DRP and about 1.07×10¹² DRP.

19. A method of treating a complement disorder comprising contacting a cell with a therapeutically effective amount of a pharmaceutical composition having as an active agent a nucleic acid encoding a human sCD59 protein or a source of expression of a human sCD59 protein comprising administering the pharmaceutical composition to a subject in need thereof.

20. The method according to claim 19, wherein the complement disorder is GA.

21. The method according to claim 19, wherein the contacting is by intravitreal injection.

22. The method according to claim 21, wherein the intravitreal injection is a single injection.

23. The method according to claim 19, wherein the affected cell is a retinal cell.

24. The method according to claim 19, wherein the therapeutically effective amount is a dose of viral particles selected from the group consisting of about 3.56×10¹⁰ DNAse-resistant particles (DRP), about 1.071×10¹¹ DRP, about 3.56×10¹¹ DRP and about 1.07×10¹² DRP.
